(19) United States
(12) Patent Application Publication
(10) Pub. No.: US 2021/0128531 A1
(43) Pub. Date: May 6, 2021

(54) STABLE COMPOSITION INCLUDING ANGIOTENSIN II, PREPARATION METHOD AND APPLICATION METHOD THEREOF

(71) Applicant: NANJING HAIWEI PHARMACEUTICAL TECHNOLOGIES CO., LTD., Nanjing (CN)

(72) Inventors: Likun WANG, Nanjing (CN); Fenge ZHANG, Nanjing (CN); Guoxiang GU, Nanjing (CN)

(73) Assignee: NANJING HAIWEI PHARMACEUTICAL TECHNOLOGIES CO., LTD., Nanjing (CN)

(21) Appl. No.: 16/844,976

(22) Filed: Apr. 9, 2020

(30) Foreign Application Priority Data
Oct. 31, 2019 (CN) 201911054867.4

(51) Int. Cl. A61K 31/4178; A61K 47/40; A61K 47/26; A61K 47/42

(52) U.S. Cl. CPC A61K 31/4178; A61K 47/42; A61K 47/26; A61K 47/40

(57) ABSTRACT

The present disclosure relates to a field of compounds, and more particularly, a stable composition including angiotensin II, a preparation method, and an application method thereof. The composition may include: the angiotensin II; and an excipient, including one or more substances selected from a sugar, an alcohol, a polymer, a surfactant, or an amino acid. The present disclosure may be used for the stable composition including the angiotensin II with high stability, and may be stored under room temperature for more than two years, which solves a problem that the poor stability of the angiotensin II requires cold chain transportation and storage, thereby greatly improving the security and effectiveness of the medicine.

### CROSS-REFERENCE TO RELATED APPLICATIONS

This application claims priority of Chinese Patent Application No. 201911054867.4 filed on Oct. 31, 2019, the entire contents of which are incorporated herein by reference.

### TECHNICAL FIELD

The present disclosure relates to a field of compounds, and more particularly, a stable composition including angiotensin II, a preparation method, and an application method thereof.

### BACKGROUND

Angiotensin II is the main active substance of the renin-angiotensin system (RAS). It plays a key role in the acute and chronic regulation of systemic arterial blood pressure. The angiotensin II has been used to treat septic shock, acute kidney injury, cardiac arrest, severe and refractory hypotension. However, the current drug product(s) including the angiotensin II has poor stability and need to be refrigerated during transportation and storage. The poor stability of current drug product(s) may lead to safety and efficacy issues as well as undesirable shelf-life. In addition, refrigeration storage requirement of current drug product(s) containing angiotensin II may cause inconvenience when using the drug(s) as a number of indication(s) of angiotensin II are under emergency situations, under which, refrigeration storage condition might not available. Therefore, it is desirable to develop a more stable composition including the angiotensin II, allowing better usage convenience and availability.

### SUMMARY

The purpose of the present disclosure is to provide a stable composition including angiotensin II, a preparation method, and an application method thereof, to improve stability of drug product(s) containing angiotensin II, allowing room temperature storage, and extending product shelf-life thereof.

An aspect of the present disclosure provides a stable composition including the angiotensin II, includes: angiotensin II; and one or more excipient(s) selected from the following substance(s): sugar, alcohol, polymer, surfactant and/or amino acid.

Another aspect of the present disclosure provides a preparation method of the composition, and the method includes: dissolving the angiotensin II and the excipient(s) in a solvent to obtain a solution, wherein the solvent is selected from one or more of the following: water, acetonitrile, N-methylpyrrolidone, dimethyl sulfoxide, dichloromethane, acetone, or alcohol; and solidifying the solution to obtain the composition.

Still another aspect of the present disclosure provides a method for using the composition, and the method includes: storing the composition in the powder chamber of a dual chamber bag, and an isotonic solution for infusion in the liquid chamber of the dual chamber bag, wherein the powder chamber is isolated from the liquid chamber by a peelable seal. Squeezing the liquid chamber bag may potentially detach the peelable seal to allow mixing of powder and isotonic solution for infusion usage.

Still another aspect of the present disclosure provides a method for using the composition, and the method includes: storing the composition in a medical container, which at least including a sealed vial; adding a medical liquid to the medical container to dissolve the composition; and using the obtained solution for infusion.

### DETAILED DESCRIPTION

As used in the disclosure and the appended claims, the singular forms “a,” “an,” and “the” may be intended to include the plural forms as well, unless the context clearly indicates otherwise. In general, the terms “comprise” and “include” merely prompt to include steps and elements that have been clearly identified, and these steps and elements do not constitute an exclusive listing. The methods or devices may also include other steps or elements. The range of values used for the present disclosure is for the purpose of concisely describing each value included in the range.

The existing medical composition(s) including angiotensin II is(are) generally in a form of solution. And the composition(s) was(were) usually needs to be stored under refrigeration in an environment of 2° C. to 8° C. It's a rigorous demand of storing the drug(s). The medical stable composition disclosed in the present disclosure may be stored under a less strict storage condition, such as a room temperature condition. The shelf-life of the disclosed stable composition may be longer than two years.

The stable composition including the angiotensin II disclosed in the present disclosure may include the angiotensin II and an excipient. The chemical name of the angiotensin II involved in the present disclosure is L-aspartyl-L-arginyl-L-valyl-L-tyrosyl-L-isoleucine-L-histidyl-L-prolyl-L-phenyalanine. The molecular formula of the angiotensin II is C₅₀H₇₁N₁₃O₁₂, and the structural formula is as follows:

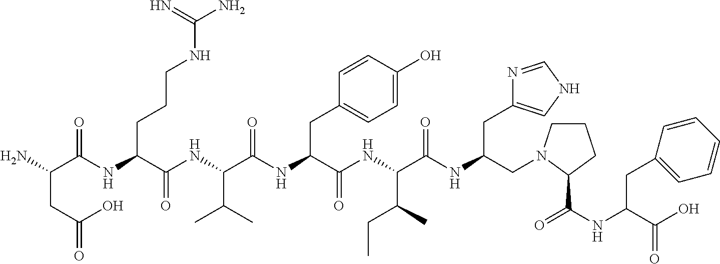 [SMILES: CC[C@H](C)[C@H](NC(=O)[C@H](Cc1ccc(O)cc1)NC(=O)[C@@H](NC(=O)[C@H](CCCNC(=N)N)NC(=O)[C@@H](N)CC(=O)O)C(C)C)C(=O)N[C@@H](Cc1c[nH]cn1)CN1CCC[C@H]1C(=O)NC(Cc1ccccc1)C(=O)O]

The corresponding amino acid sequence of the angiotensin II may be represented as Asp-Arg-Val-Tyr-Ile-His-Pro-Phe. Other analogs of the angiotensin II with the amino acid sequences Asp-Arg-Val-Tyr-Val-His-Pro-Phe, Asn-Arg-Val-Phe-Ile-His-Pro-Phe, Val-Tyr-Ile-His-Pro-Phe, Asn-Arg-Val-Tyr-Tyr-Val-His-Pro-Phe, Asn-Arg-Val-Tyr-Ile-His-Pro-Ile, and Asn-Arg-Val-Tyr-Ile-His-Pro-Ala, may also be used as the angiotensin II in the present disclosure. The term “angiotensin II” as used in the present disclosure, without further specificity, is intended to refer to any of the above various forms, as well as combinations thereof.

The sequence of the angiotensin II used in the composition and method disclosed in the present disclosure may be homologous to the sequence of the angiotensin II described above. In some embodiments, the present disclosure may include an isolated or recombinant amino acid sequence that is at least 80%, 85%, 90%, 95%, 97%, 98%, 99%, or 100% identical to the amino acid sequence described above. Any such variant sequences may be used instead of the angiotensin II as described in the preceding paragraph.

Similarly, the angiotensin II may be any suitable pharmaceutically acceptable salt, protonated form, deprotonated form, acetylated form, deacetylated form, and/or prodrug form of the peptides with the amino acid sequence described above. Exemplary molecular formula of the pharmaceutically acceptable salt of the angiotensin II may be presented as: C₅₀H₇₁N₁₃O₁₂—(C₂H₄O₂)ₙ, wherein “n” may be an integer greater than 0 (such as 1, 2, 3, etc.), and “n” may be the number of molecules of acetic acid, theoretically n=3. The structural formula of the pharmaceutically acceptable salt of the angiotensin II may be shown as follow:

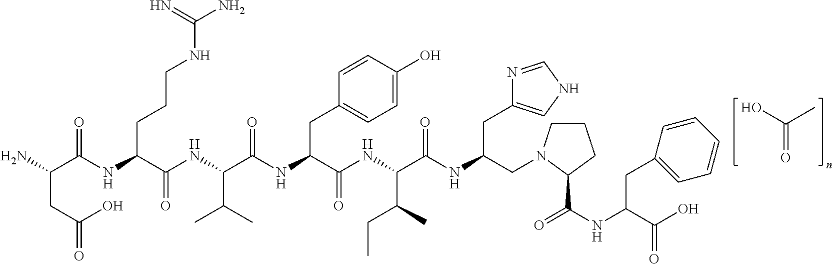

The angiotensin II used in the present disclosure may be prepared in laboratory, or obtained readily from commercial sources.

A mass ratio of the angiotensin II to the excipient in the stable composition including the angiotensin II described in the present disclosure may be no more than 10:10. In some embodiments, the mass ratio of the angiotensin II to the excipient may be no more than 9:10. In some embodiments, the mass ratio of the angiotensin II to the excipient may be no more than 8:10. In some embodiments, the mass ratio of the angiotensin II to the excipient may be no more than 7:10. In some embodiments, the mass ratio of the angiotensin II to the excipient may be no more than 6:10. In some embodiments, the mass ratio of the angiotensin II to the excipient may be no more than 5:10. In some embodiments, the mass ratio of the angiotensin II to the excipient may be no more than 4:10. In some embodiments, the mass ratio of the angiotensin II to the excipient may be no more than 3:10. In some embodiments, the mass ratio of the angiotensin II to the excipient may be no more than 2:10. In some embodiments, the mass ratio of the angiotensin II to the excipient may be no more than 1:10. In some embodiments, the mass ratio of the angiotensin II to the excipient may be no more than 0.9:10. In some embodiments, the mass ratio of the angiotensin II to the excipient may be no more than 0.8:10. In some embodiments, the mass ratio of the angiotensin II to the excipient may be no more than 0.7:10. In some embodiments, the mass ratio of the angiotensin II to the excipient may be no more than 0.6:10. In some embodiments, the mass ratio of the angiotensin II to the excipient may be no more than 0.5:10. In some embodiments, the mass ratio of the angiotensin II to the excipient may be no more than 0.4:10. In some embodiments, the mass ratio of the angiotensin II to the excipient may be no more than 0.3:10.

The stable composition including the angiotensin II in the present disclosure may include an excipient. The excipient may improve the stability of the composition prepared by the angiotensin II. In some embodiments, the excipient may include one or more of a sugar, an alcohol, a polymer, a surfactant, or an amino acid. Exemplary sugars may include one or more of sucrose, trehalose, galactose, lactose, glucose, raffinose, mannose, fructose, maltose, ribose, xylose, or the like. Exemplary alcohols may include one or more of sorbitol, inositol, mannitol, tert-butanol, or xylitol. The said alcohol contains one or more hydroxyl groups, e.g., ethanol, polyol. In some embodiments, the alcohol may be polyol. Exemplary polymers may include one or more of hydroxyethyl starch, dextran, polysucrose, Arabic gum, gel, cellulose, methylcellulose, pectin, PVP (polyvinylpyrrolidone), VitE TPGS (vitamin E polyethylene glycol succinate), PEG300, PEG400, other molecular weights of PEG, povidone, human albumin, bovine serum albumin, maltodextrin, hydroxypropyl-β-cyclodextrin, sulfobutyl-β-cyclodextrin, or other cyclodextrins. Exemplary surfactants may include one or more of Tween 20, Tween 80, poloxamer (polyoxyethylene-polyoxypropylene ether block copolymer), acetonitrile, transcutol (diethylene glycol monoethyl ether), or HS-15 (lithium polyethylene glycol (PEG) dodecyl stearate). Exemplary amino acids may include one or more of proline, 4-hydroxyproline, glycine, arginine, L-serine, β-alanine, sarcosine, lysine, arginine, histidine, glutamic acid, aspartic acid, or malic acid. In some embodiments, the excipient may also include other substances, for example, vitamin C, vitamin D, vitamin E, lecithin, D(−)-isoascorbic acid, sodium L-ascorbate, sodium thiosulfate, butylated hydroxyl anisole, dibutylate hydroxytoluene, propyl gallate, disodiurn ethylenediamine tetraacetic acid, thiourea, and protein hydrolysate, which may be used for antioxidant effects, soft candy, gelatin, etc., and salts such as sodium sulfate, calcium lactate, sodium glutamate, sodium chloride, potassium chloride, ammonium acetate, ammonium chloride, or the like, which may be used for filling.

In some embodiments, the composition may also include a buffer agent. The buffer agent may provide a suitable pH range to lyophilize during the preparation of the composition (e.g., lyophilization to prepare the composition). For example, in some embodiments, the buffer agent may contain from 0.01% to 5% by weight of the composition. In some embodiments, the buffer agent may contain from 0.1% to 4.5% by weight of the composition. In some embodiments, the buffer agent may contain from 0.2% to 4.0% by weight of the composition. In some embodiments, the buffer agent may contain from 0.3% to 3.5% by weight of the composition. In some embodiments, the buffer agent may contain from 0.4% to 3.0% by weight of the composition. In some embodiments, the buffer agent may contain from 0.5% to 2.5% by weight of the composition. In some embodiments, the buffer agent may contain from 0.6% to 2.0% by weight of the composition. In some embodiments, the buffer agent may contain from 0.7% to 1.5% by weight of the composition. In some embodiments, the buffer agent may contain from 0.8% to 1.2% by weight of the composition. In some embodiments, the buffer agent may contain from 0.9% to 1.1% by weight of the composition. In some embodiments, the buffer agent may contain 1.0% by weight of the composition. In some embodiments, the buffer agent may provide the pH range of 4.0-9.0. In some embodiments, the buffer agent may provide the pH range of 4.5-8.5. In some embodiments, the buffer agent may provide the pH range of 5.0 to 8.0. In some embodiments, the buffer agent may provide the pH range of 5.5-7.5. In some embodiments, the buffer agent may provide the pH range of 6.0-7.0. In some embodiments, the buffer agent may provide the pH range of 6.5. Exemplary buffer agents may include one or more of acetic acid, phosphoric acid, citric acid, tartaric acid, EDTA, amino acids, Tris (trihydromethylaminomethane), sodium hydroxide, phosphate, and sodium hydrogen phosphate.

In some embodiments, the composition stored under a first environmental condition after completion of preparation, a reduction value of a purity of the angiotensin II after a first time period may be less than a first threshold. An increment of a first impurity associated with the angiotensin II may be less than a second threshold. In some embodiments, the purity refers to the ratio of components to the sum of the components themselves and related impurities (excluding excipients). For example, the purity of the angiotensin II refers to the ratio of the angiotensin II to the sum of the angiotensin II and the angiotensin II related impurities.

In some embodiments, the first threshold may be 3%. Alternatively, the first threshold may be 3.1%. Alternatively, the first threshold may be 3.2%. Alternatively, the first threshold may be 3.3%. Alternatively, the first threshold may be 3.4%. Alternatively, the first threshold may be 3.5%. Alternatively, the first threshold may be 3.6%. Alternatively, the first threshold may be 3.8%. Alternatively, the first threshold may be 3.8%. Alternatively, the first threshold may be 3.9%. Alternatively, the first threshold may be 4%. Alternatively, the first threshold may be 4.1%. Alternatively, the first threshold may be 4.2%. Alternatively, the first threshold may be 4.3%. Alternatively, the first threshold may be 4.4%. Alternatively, the first threshold may be 4.5%. Alternatively, the first threshold may be 4.6%. Alternatively, the first threshold may be 4.7%. Alternatively, the first threshold may be 4.8%. Alternatively, the first threshold may be 4.9%. Alternatively, the first threshold may be 5%.

In some embodiments, the second threshold may be 0.5%. Alternatively, the second threshold may be 0.51%. Alternatively, the second threshold may be 0.52%. Alternatively, the second threshold may be 0.53%. Alternatively, the second threshold may be 0.54%. Alternatively, the second threshold may be 0.55%. Alternatively, the second threshold may be 0.56%. Alternatively, the second threshold may be 0.57%. Alternatively, the second threshold may be 0.58%. Alternatively, the second threshold may be 0.59%. Alternatively, the second threshold may be 0.60%. Alternatively, the second threshold may be 0.61%. Alternatively, the second threshold may be 0.62%. Alternatively, the second threshold may be 0.63%. Alternatively, the second threshold may be 0.64%. Alternatively, the second threshold may be 0.65%. Alternatively, the second threshold may be 0.66%. Alternatively, the second threshold may be 0.67%. Alternatively, the second threshold may be 0.68%. Alternatively, the second threshold may be 0.69%. Alternatively, the second threshold may be 0.7%.

In some embodiments, the first environment temperature may refer to an environment temperature in which the composition is in direct contact, and may also be a packaging temperature at which the composition is packaged. In some embodiments, the first environment temperature may be no more than 40° C. Alternatively, the first environment temperature may be no more than 39° C. Alternatively, the first environment temperature may be no more than 38° C. Alternatively, the first environment temperature may be no more than 37° C. Alternatively, the first environment temperature may be no more than 36° C. Alternatively, the first environment temperature may be no more than 35° C. Alternatively, the first environment temperature may be no more than 34° C. Alternatively, the first environment temperature may be no more than 33° C. Alternatively, the first environment temperature may be no more than 32° C. Alternatively, the first environment temperature may be no more than 31° C. Alternatively, the first environment temperature may be no more than 30° C. In some embodiments, the composition stored under the first environment relative humidity may be 30%-80%. The first environment relative humidity may refer to the relative humidity of the environment that the composition is exposed to during storage, including the environment in which the composition is in direct contact, and after packaged. Alternatively, the first environment relative humidity may be between 35% and 75%. Alternatively, the first environment relative humidity may be 40%-70%. Alternatively, the first environment relative humidity may be 45%-65%. Alternatively, the first environment relative humidity may be 50%-60%. Alternatively, the first environment relative humidity may be 60%. In some embodiments, the first time period may be at least two years. Here, the term “at least” may mean a time range of 0 to 2 years, and more than two years. The “at least” in the following description is expressed in a similar time range. In some embodiments, the first time period may be at least two years and two months. In some embodiments, the first time period may be at least two years and four months. In some embodiments, the first time period may be at least two years and six months. In some embodiments, the first time period may be at least two years and eight months. In some embodiments, the first time period may be at least two years and ten months. In some embodiments, the first time period may be at least three years.

The present disclosure also provides a method for using the stable composition described above, the method may include: storing the composition in a powder chamber of a dual chamber container. The liquid chamber of the dual-chamber bag may store a medical liquid. In some embodiments, the medical liquid may include physiological saline, a glucose solution, or other solution(s) suitable for infusion. The concentration of the physiological saline may be 0.9%. The concentration of the glucose solution may be 5%. The powder chamber may be isolated from the liquid chamber by a peelable seal. When infusion solution containing angiotensin II is needed, the liquid chamber bag may be squeezed to open the peelable seal to mix the composition with the medical liquid to form a ready-to-use infusion solution. The drug product described in the present disclosure may not need any liquid transfer operation to prepare liquid for infusion. The disclosed dual-chamber system may yield ready-to-use infusion liquid after direct squeezing the liquid chamber and reconstitution, allowing faster drug infusion solution preparation, which is better suited for first aid purposes of angiotensin II.

The present disclosure also provides a method for preparing the stable composition described above, the method may include: dissolving the angiotensin II and the excipient in a solvent to obtain a solution. In some embodiments, the solvent may be selected from one or more of water, acetonitrile, N-methylpyrrolidone, dimethyl sulfoxide, dichloromethane, acetone or alcohol. In some embodiments, the alcohol may include one or more of ethanol, isopropanol, propanediol, glycerol, and tert-butanol. Solid composition may then be obtained by removing solvents. The solidifying method(s) may include freeze drying or spray drying. The composition preparation process in the present disclosure is scalable.

In some embodiments, the angiotensin II and the excipient may be the same or similar to the description above, and not be repeated herein. In some embodiments, a mass ratio of the angiotensin II to the excipient in the stable composition including the angiotensin II described in the present disclosure may be no more than 10:10. In some embodiments, the mass ratio of the angiotensin II to the excipient may be no more than 9:10. In some embodiments, the mass ratio of the angiotensin II to the excipient may be no more than 8:10. In some embodiments, the mass ratio of the angiotensin II to the excipient may be no more than 7:10. In some embodiments, the mass ratio of the angiotensin II to the excipient may be no more than 6:10. In some embodiments, the mass ratio of the angiotensin II to the excipient may be no more than 5:10. In some embodiments, the mass ratio of the angiotensin II to the excipient may be no more than 4:10. In some embodiments, the mass ratio of the angiotensin II to the excipient may be no more than 3:10. In some embodiments, the mass ratio of the angiotensin II to the excipient may be no more than 2:10. In some embodiments, the mass ratio of the angiotensin II to the excipient may be no more than 1:10. In some embodiments, the mass ratio of the angiotensin II to the excipient may be no more than 0.9:10. In some embodiments, the mass ratio of the angiotensin II to the excipient may be no more than 0.8:10. In some embodiments, the mass ratio of the angiotensin II to the excipient may be no more than 0.7:10. In some embodiments, the mass ratio of the angiotensin II to the excipient may be no more than 0.6:10. In some embodiments, the mass ratio of the angiotensin II to the excipient may be no more than 0.5:10. In some embodiments, the mass ratio of the angiotensin II to the excipient may be no more than 0.4:10. In some embodiments, the mass ratio of the angiotensin II to the excipient may be no more than 0.3:10.

In some embodiments, the composition stored under a first environmental condition after completion of preparation, a reduction value of a purity of the angiotensin II after a first time period may be less than a first threshold. An increment of a first impurity associated with the angiotensin II may be less than a second threshold. In some embodiments, the purity refers to the ratio of components to the sum of the components themselves and related impurities (excluding excipients). For example, the purity of the angiotensin II refers to the ratio of the angiotensin II to the sum of the angiotensin II and the angiotensin II related impurities.

In some embodiments, the first threshold may be 3%. Alternatively, the first threshold may be 3.1%. Alternatively, the first threshold may be 3.2%. Alternatively, the first threshold may be 3.3%. Alternatively, the first threshold may be 3.4%. Alternatively, the first threshold may be 3.5%. Alternatively, the first threshold may be 3.6%. Alternatively, the first threshold may be 3.8%. Alternatively, the first threshold may be 3.8%. Alternatively, the first threshold may be 3.9%. Alternatively, the first threshold may be 4%. Alternatively, the first threshold may be 4.1%. Alternatively, the first threshold may be 4.2%. Alternatively, the first threshold may be 4.3%. Alternatively, the first threshold may be 4.4%. Alternatively, the first threshold may be 4.5%. Alternatively, the first threshold may be 4.6%. Alternatively, the first threshold may be 4.7%. Alternatively, the first threshold may be 4.8%. Alternatively, the first threshold may be 4.9%. Alternatively, the first threshold may be 5%.

In some embodiments, the second threshold may be 0.5%. Alternatively, the second threshold may be 0.51%. Alternatively, the second threshold may be 0.52%. Alternatively, the second threshold may be 0.53%. Alternatively, the second threshold may be 0.54%. Alternatively, the second threshold may be 0.55%. Alternatively, the second threshold may be 0.56%. Alternatively, the second threshold may be 0.57%. Alternatively, the second threshold may be 0.58%. Alternatively, the second threshold may be 0.59%. Alternatively, the second threshold may be 0.60%. Alternatively, the second threshold may be 0.61%. Alternatively, the second threshold may be 0.62%. Alternatively, the second threshold may be 0.63%. Alternatively, the second threshold may be 0.64%. Alternatively, the second threshold may be 0.65%. Alternatively, the second threshold may be 0.66%. Alternatively, the second threshold may be 0.67%. Alternatively, the second threshold may be 0.68%. Alternatively, the second threshold may be 0.69%. Alternatively, the second threshold may be 0.7%.

In some embodiments, the first environment temperature may refer to an environment temperature in which the composition is in direct contact, and may also be a packaging temperature at which the composition is packaged. In some embodiments, the first environment temperature may be no more than 40° C. Alternatively, the first environment temperature may be no more than 39° C. Alternatively, the first environment temperature may be no more than 38° C. Alternatively, the first environment temperature may be no more than 37° C. Alternatively, the first environment temperature may be no more than 36° C. Alternatively, the first environment temperature may be no more than 35° C. Alternatively, the first environment temperature may be no more than 34° C. Alternatively, the first environment temperature may be no more than 33° C. Alternatively, the first environment temperature may be no more than 32° C. Alternatively, the first environment temperature may be no more than 31° C. Alternatively, the first environment temperature may be no more than 30° C. In some embodiments, the composition stored under the first environment relative humidity may be 30%-80%. The first environment relative humidity may refer to the relative humidity of the environment that the composition is exposed to during storage, including the environment in which the composition is in direct contact, and after packaged. Alternatively, the first environment relative humidity may be between 35% and 75%. Alternatively, the first environment relative humidity may be 40%-70%. Alternatively, the first environment relative humidity may be 45%-65%. Alternatively, the first environment relative humidity may be 50%-60%. Alternatively, the first environment relative humidity may be 60%. In some embodiments, the first time period may be at least two years. Here, the term “at least” may mean a time range of 0 to 2 years, and more than two years. The “at least” in the following description is expressed in a similar time range. In some embodiments, the first time period may be at least two years and two months. In some embodiments, the first time period may be at least two years and four months. In some embodiments, the first time period may be at least two years and six months. In some embodiments, the first time period may be at least two years and eight months. In some embodiments, the first time period may be at least two years and ten months. In some embodiments, the first time period may be at least three years.

The present disclosure also provides a method for using the stable composition described above. In some embodiments, the composition may be stored in a medical container, including a sealed vial (for example, a penicillin bottle), a cassette bottle, an ampoule, or the like. In the application, a predetermined amount of the medical liquid may be added to the medical container. The medical liquid may include a physiological saline, a glucose solution, or other solution(s) suitable for infusion. After mixing, the composition may be reconstituted for therapeutic operations such as injection, infusion, or the like. For example, a syringe may be used to draw a medical liquid which dissolves the composition to inject directly into the patient, or reconstituted solution may be further mixed with other medical solution for infusion purpose.

The present disclosure also provides a method for preparing the stable composition described above, the method may include: dissolving the angiotensin II and the excipient in a solvent to obtain a solution. In some embodiments, the solvent may be selected from one or more of water, acetonitrile, N-methylpyrrolidone, dimethyl sulfoxide, dichloromethane, acetone or alcohol. In some embodiments, the alcohol may include one or more of ethanol, isopropanol, propanediol, glycerol, and tert-butanol. Solid composition may then be obtained by removing solvents. The solidifying method(s) may include freeze drying or spray drying. The composition preparation process in the present disclosure is scalable.

In some embodiments, the angiotensin II and the excipient may be the same or similar to the description above, and not be repeated herein. In some embodiments, a mass ratio of the angiotensin II to the excipient in the stable composition including the angiotensin II described in the present disclosure may be no more than 10:10. In some embodiments, the mass ratio of the angiotensin II to the excipient may be no more than 9:10. In some embodiments, the mass ratio of the angiotensin II to the excipient may be no more than 8:10. In some embodiments, the mass ratio of the angiotensin II to the excipient may be no more than 7:10. In some embodiments, the mass ratio of the angiotensin II to the excipient may be no more than 6:10. In some embodiments, the mass ratio of the angiotensin II to the excipient may be no more than 5:10. In some embodiments, the mass ratio of the angiotensin II to the excipient may be no more than 4:10. In some embodiments, the mass ratio of the angiotensin II to the excipient may be no more than 3:10. In some embodiments, the mass ratio of the angiotensin II to the excipient may be no more than 2:10. In some embodiments, the mass ratio of the angiotensin II to the excipient may be no more than 1:10. In some embodiments, the mass ratio of the angiotensin II to the excipient may be no more than 0.9:10. In some embodiments, the mass ratio of the angiotensin II to the excipient may be no more than 0.8:10. In some embodiments, the mass ratio of the angiotensin II to the excipient may be no more than 0.7:10. In some embodiments, the mass ratio of the angiotensin II to the excipient may be no more than 0.6:10. In some embodiments, the mass ratio of the angiotensin II to the excipient may be no more than 0.5:10. In some embodiments, the mass ratio of the angiotensin II to the excipient may be no more than 0.4:10. In some embodiments, the mass ratio of the angiotensin II to the excipient may be no more than 0.3:10.

In some embodiments, the composition stored under a first environmental condition after completion of preparation, a reduction value of a purity of the angiotensin II after a first time period may be less than a first threshold. An increment of a first impurity associated with the angiotensin II may be less than a second threshold. In some embodiments, the purity refers to the ratio of components to the sum of the components themselves and related impurities (excluding excipients). For example, the purity of the angiotensin II refers to the ratio of the angiotensin II to the sum of the angiotensin II and the angiotensin II related impurities.

In some embodiments, the first threshold may be 3%. Alternatively, the first threshold may be 3.1%. Alternatively, the first threshold may be 3.2%. Alternatively, the first threshold may be 3.3%. Alternatively, the first threshold may be 3.4%. Alternatively, the first threshold may be 3.5%. Alternatively, the first threshold may be 3.6%. Alternatively, the first threshold may be 3.8%. Alternatively, the first threshold may be 3.8%. Alternatively, the first threshold may be 3.9%. Alternatively, the first threshold may be 4%. Alternatively, the first threshold may be 4.1%. Alternatively, the first threshold may be 4.2%. Alternatively, the first threshold may be 4.3%. Alternatively, the first threshold may be 4.4%. Alternatively, the first threshold may be 4.5%. Alternatively, the first threshold may be 4.6%. Alternatively, the first threshold may be 4.7%. Alternatively, the first threshold may be 4.8%. Alternatively, the first threshold may be 4.9%. Alternatively, the first threshold may be 5%.

In some embodiments, the second threshold may be 0.5%. Alternatively, the second threshold may be 0.51%. Alternatively, the second threshold may be 0.52%. Alternatively, the second threshold may be 0.53%. Alternatively, the second threshold may be 0.54%. Alternatively, the second threshold may be 0.55%. Alternatively, the second threshold may be 0.56%. Alternatively, the second threshold may be 0.57%. Alternatively, the second threshold may be 0.58%. Alternatively, the second threshold may be 0.59%. Alternatively, the second threshold may be 0.60%. Alternatively, the second threshold may be 0.61%. Alternatively, the second threshold may be 0.62%. Alternatively, the second threshold may be 0.63%. Alternatively, the second threshold may be 0.64%. Alternatively, the second threshold may be 0.65%. Alternatively, the second threshold may be 0.66%. Alternatively, the second threshold may be 0.67%. Alternatively, the second threshold may be 0.68%. Alternatively, the second threshold may be 0.69%. Alternatively, the second threshold may be 0.7%.

In some embodiments, the first environment temperature may refer to an environment temperature in which the composition is in direct contact, and may also be a packaging temperature at which the composition is packaged. In some embodiments, the first environment temperature may be no more than 40° C. Alternatively, the first environment temperature may be no more than 39° C. Alternatively, the first environment temperature may be no more than 38° C. Alternatively, the first environment temperature may be no more than 37° C. Alternatively, the first environment temperature may be no more than 36° C. Alternatively, the first environment temperature may be no more than 35° C. Alternatively, the first environment temperature may be no more than 34° C. Alternatively, the first environment temperature may be no more than 33° C. Alternatively, the first environment temperature may be no more than 32° C. Alternatively, the first environment temperature may be no more than 31° C. Alternatively, the first environment temperature may be no more than 30° C. In some embodiments, the composition stored under the first environment relative humidity may be 30%-80%. The first environment relative humidity may refer to the relative humidity of the environment that the composition is exposed to during storage, including the environment in which the composition is in direct contact, and after packaged. Alternatively, the first environment relative humidity may be between 35% and 75%. Alternatively, the first environment relative humidity may be 40%-70%. Alternatively, the first environment relative humidity may be 45%-65%. Alternatively, the first environment relative humidity may be 50%-60%. Alternatively, the first environment relative humidity may be 60%. In some embodiments, the first time period may be at least two years. Here, the term “at least” may mean a time range of 0 to 2 years, and more than two years. The “at least” in the following description is expressed in a similar time range. In some embodiments, the first time period may be at least two years and two months. In some embodiments, the first time period may be at least two years and four months. In some embodiments, the first time period may be at least two years and six months. In some embodiments, the first time period may be at least two years and eight months. In some embodiments, the first time period may be at least two years and ten months. In some embodiments, the first time period may be at least three years.

### Embodiments

The present disclosure is further described below in different embodiments.

### Embodiment 1—Preparation of Composition A1

The angiotensin II and the excipient mannitol were weighed and dissolved in pure water to obtain a clear solution. The mass ratio of the two (the angiotensin II:the excipient mannitol) was 0.3:10. The clear solution was freeze-dried to obtain the composition A1.

### Embodiment 2-Preparation of Composition A2

The angiotensin II and the excipient sulfobutyl-β-cyclodextrin were weighed and dissolved in pure water to obtain a clear solution. The mass ratio of the two (the angiotensin II:the excipient sulfobutyl-β-cyclodextrin) was 0.3:10. The clear solution was freeze-dried to obtain the composition A2.

### Embodiment 3—Preparation of Composition A3

The angiotensin II and the excipient lactose were weighed and dissolved in pure water to obtain a clear solution. The mass ratio of the two (the angiotensin II:the excipient lactose) was 0.3:5. The clear solution was freeze-dried to obtain the composition A3.

### Embodiment 4—Preparation of Composition A5

The angiotensin II and the excipient maltose were weighed and dissolved in pure water to obtain a clear solution. The mass ratio of the two (the angiotensin II:the excipient maltose) was 0.3:10. The clear solution was freeze-dried to obtain the composition A5.

### Embodiment 5—Preparation of Composition A6

The angiotensin II and the excipient sucrose were weighed and dissolved in pure water to obtain a clear solution. The mass ratio of the two (the angiotensin II:the excipient sucrose) was 0.3:10. The clear solution was freeze-dried to obtain the composition A6

### Embodiment 6—Preparation of Composition A7

The angiotensin II and the excipient trehalose were weighed and dissolved in pure water to obtain a clear solution. The mass ratio of the two (the angiotensin II:the excipient trehalose) was 0.3:10. The clarified solution was freeze-dried to obtain the composition A7.

### Embodiment 7—Preparation of Composition A9

The angiotensin II and the excipient inositol were weighed and dissolved in pure water to obtain a clear solution. The mass ratio of the two (the angiotensin II:the excipient inositol) was 0.3:5. The clear solution was freeze-dried to obtain composition A9.

### Embodiment 8—Preparation of Composition A10

The angiotensin II and the excipient human albumin were weighed and dissolved in pure water to obtain a clear solution. The mass ratio of the two (the angiotensin II:the excipient human albumin) was 0.3:5. The clear solution was freeze-dried to obtain the composition A10.

### Embodiment 9—Preparation of Composition A11

The angiotensin II and the excipient glycine were weighed and dissolved in pure water to obtain a clear solution. The mass ratio of the two (the angiotensin II:the excipient glycine) of the two was 0.3:5. The clear solution was freeze-dried to obtain the composition A11.

### Embodiment 10—Preparation of Composition A12

The angiotensin II and the excipient hydroxyethyl starch were weighed and dissolved in pure water to obtain a clear solution. The mass ratio of the two (the angiotensin II:the excipient hydroxyethyl starch) was 0.3:5. The clear solution was freeze-dried to obtain the composition A12.

### Embodiment 11—Preparation of Composition A13

The angiotensin II and the excipient povidone were weighed and dissolved in pure water to obtain a clear solution. The mass ratio of the two (the angiotensin II:the excipient povidone) was 0.3:10. The clear solution was freeze-dried to obtain the composition A13.

### Embodiment 12—Preparation of Composition A14

The angiotensin II and the excipient dextran were dissolved in pure water to obtain a clear solution. The mass ratio of the two (the angiotensin II:the excipient dextran) was 0.3:5. The clear solution was freeze-dried to obtain the composition A14.

### Embodiment 13—Purity Test

The compositions prepared by the embodiment 1-the embodiment 12 were used as experimental groups, and API (i.e., drug substance) was used as the control group. The accelerated test was carried out at the test temperature and the test relative humidity, and the purity of the angiotensin II in the compositions prepared by the embodiment 1 to the embodiment 12 and the control group were tested after the test time. The purity test is based on a High Performance Liquid Chromatography (HPLC).

The HPLC method used a Shimadzu LC-16 instrument. The mobile phases were 82% v/v HPLC buffers A (A=10 mM KH₂PO₄ in water, pH=3) and 18% v/v B(B=Acetonitrile) for 30 min at flow rate 0.5 mL/min. The Waters XBridge C18 4.6×150 mm, 5 μm column was at temperature 40° C. with UV detection at 210 nm. The injection volume was 2 uL.

The test results are shown in Tables 1 to 14. The test temperatures were 0, 55, 65, 70, and 75° C., respectively. The relative humidity of the test was 0% and 11%, respectively. The test time was 0, 1, 2, 3, 6, 7, 10, 17, and 21 days, respectively. The symbol “/” in the tables denoteds “Not detected”.

TABLE 1
Test result of related substance of composition A1

| T/ | RH/ | Time/ | RRT | | | | | |
|---|---|---|---|---|---|---|---|---|
| ° C. | % | day | 0.22 | 0.25 | 0.26 | 0.31 | 0.37 | 0.48 |
| 0 | 0 | 0 | / | 0.36 | 0.33 | 0.35 | / | / |
| 0 | 0 | 0 | / | 0.35 | 0.31 | 0.34 | / | / |
| 55 | 0 | 10 | / | 0.37 | 0.35 | 0.29 | / | / |
| 55 | 0 | 21 | / | 0.35 | 0.31 | 0.42 | / | / |
| 55 | 11 | 7 | / | 0.35 | 0.34 | 0.25 | / | / |
| 55 | 11 | 21 | / | 0.46 | 0.35 | 0.22 | / | / |
| 65 | 0 | 3 | / | 0.37 | 0.35 | 0.29 | / | / |
| 65 | 0 | 21 | / | 0.41 | 0.38 | 0.24 | / | / |
| 65 | 11 | 2 | / | 0.36 | 0.34 | 0.25 | / | / |
| 65 | 11 | 17 | 0.06 | 0.41 | 0.41 | 0.21 | / | / |
| 70 | 11 | 1 | / | 0.36 | 0.37 | 0.27 | / | / |
| 70 | 11 | 10 | / | 0.42 | 0.45 | 0.24 | / | 0.02 |
| 75 | 0 | 1 | / | 0.37 | 0.33 | 0.27 | / | / |
| 75 | 0 | 7 | / | 0.41 | 0.41 | 0.23 | / | / |
| 75 | 11 | 1 | 0.55 | 0.51 | 0.40 | 0.24 | / | / |
| 75 | 11 | 6 | / | 0.43 | 0.46 | 0.25 | 0.04 | 0.02 |
| T/ | RH/ | Time/ | RRT | | | | | |
| ° C. | % | day | 0.64 | 0.75 | 0.79 | 0.88 | 1.00 | 1.10 |
| 0 | 0 | 0 | 0.09 | 0.72 | 0.23 | 0.12 | 95.57 | / |
| 0 | 0 | 0 | 0.09 | 0.73 | 0.23 | 0.12 | 95.47 | 0.11 |
| 55 | 0 | 10 | 0.29 | 0.76 | 0.23 | 0.14 | 94.68 | 0.21 |
| 55 | 0 | 21 | 0.37 | 0.76 | 0.25 | 0.16 | 95.19 | / |
| 55 | 11 | 7 | 0.26 | 0.75 | 0.24 | 0.13 | 94.99 | 0.22 |
| 55 | 11 | 21 | 0.44 | 0.76 | 0.24 | 0.14 | 94.34 | 0.24 |
| 65 | 0 | 3 | 0.25 | 0.75 | 0.23 | 0.14 | 94.50 | 0.24 |
| 65 | 0 | 21 | 0.80 | 0.77 | 0.24 | 0.17 | 93.04 | 0.25 |
| 65 | 11 | 2 | 0.30 | 0.77 | 0.24 | 0.13 | 94.87 | 0.24 |
| 65 | 11 | 17 | 0.83 | 0.81 | 0.24 | 0.14 | 93.36 | 0.24 |
| 70 | 11 | 1 | 0.29 | 0.75 | 0.24 | 0.13 | 94.96 | 0.22 |
| 70 | 11 | 10 | 0.96 | 0.83 | 0.24 | 0.14 | 92.51 | 0.24 |
| 75 | 0 | 1 | 0.28 | 0.75 | 0.23 | 0.13 | 94.80 | 0.24 |
| 75 | 0 | 7 | 1.10 | 0.81 | 0.23 | 0.19 | 92.19 | 0.24 |
| 75 | 11 | 1 | 0.38 | 0.77 | 0.24 | 0.13 | 94.56 | 0.25 |
| 75 | 11 | 6 | 1.10 | 0.84 | 0.25 | 0.16 | 92.84 | / |
| T/ | RH/ | Time/ | RRT | | | | | |
| ° C. | % | day | 1.17 | 1.27 | 1.59 | 1.91 | 2.12 | 2.20 |
| 0 | 0 | 0 | 2.17 | 0.06 | / | / | / | / |
| 0 | 0 | 0 | 2.18 | 0.06 | / | / | / | / |
| 55 | 0 | 10 | 2.31 | 0.05 | 0.08 | 0.09 | 0.15 | / |
| 55 | 0 | 21 | 2.13 | 0.05 | / | / | / | / |
| 55 | 11 | 7 | 2.36 | 0.06 | 0.06 | / | / | / |
| 55 | 11 | 21 | 2.32 | 0.05 | 0.17 | 0.11 | 0.14 | / |
| 65 | 0 | 3 | 2.32 | 0.05 | 0.09 | / | 0.41 | / |
| 65 | 0 | 21 | 2.29 | 0.04 | 0.72 | 0.32 | 0.33 | / |
| 65 | 11 | 2 | 2.38 | 0.06 | / | / | / | 0.43 |
| 65 | 11 | 17 | 2.39 | 0.05 | 0.51 | 0.34 | / | / |
| 70 | 11 | 1 | 2.36 | 0.06 | / | / | / | / |
| 70 | 11 | 10 | 2.37 | 0.05 | 0.60 | 0.76 | 0.13 | 0.05 |
| 75 | 0 | 1 | 2.35 | 0.06 | 0.09 | / | 0.10 | / |
| 75 | 0 | 7 | 2.30 | 0.04 | 1.43 | 0.21 | 0.11 | 0.09 |
| 75 | 11 | 1 | 2.39 | 0.05 | 0.08 | / | / | / |
| 75 | 11 | 6 | 2.18 | 0.04 | 0.78 | 0.62 | / | / |

TABLE 2
Test result of related substance of composition A2

| T/ | RH/ | Time/ | RRT | | | | | | | |
|---|---|---|---|---|---|---|---|---|---|---|
| ° C. | % | day | 0.22 | 0.26 | 0.29 | 0.31 | 0.37 | 0.62 | 0.75 | 0.78 |
| 0 | 0 | 0 | / | 0.09 | / | 0.35 | / | 0.07 | 0.77 | 0.23 |
| 0 | 0 | 0 | 0.39 | / | / | 0.34 | / | 0.07 | 0.78 | 0.23 |
| 55 | 0 | 10 | / | 0.39 | / | 0.34 | / | 0.07 | 0.77 | 0.23 |
| 55 | 0 | 21 | 0.08 | 0.47 | 0.17 | 0.37 | / | 0.07 | 0.77 | 0.23 |
| 55 | 11 | 7 | / | 0.41 | / | 0.34 | / | 0.07 | 0.78 | 0.23 |
| 55 | 11 | 21 | 0.04 | 0.44 | / | 0.34 | / | 0.08 | 0.79 | 0.22 |
| 65 | 0 | 3 | 0.02 | 0.41 | / | 0.35 | / | 0.07 | 0.77 | 0.22 |
| 65 | 0 | 21 | 0.10 | 0.51 | / | 0.36 | / | 0.08 | 0.80 | 0.23 |
| 65 | 11 | 2 | 0.38 | / | / | 0.34 | / | 0.07 | 0.78 | 0.23 |
| 65 | 11 | 17 | 0.04 | 0.46 | / | 0.35 | / | 0.07 | 0.86 | 0.22 |
| 70 | 11 | 1 | / | 0.38 | / | 0.34 | / | 0.07 | 0.78 | 0.23 |
| 70 | 11 | 10 | 0.05 | 0.46 | / | 0.34 | 0.03 | 0.07 | 0.89 | 0.23 |
| 75 | 0 | 1 | / | 0.40 | / | 0.34 | / | 0.07 | 0.78 | 0.23 |
| 75 | 0 | 7 | 0.08 | 0.51 | / | 0.37 | / | 0.07 | 0.82 | 0.23 |
| 75 | 11 | 1 | 0.03 | 0.41 | / | 0.34 | / | 0.07 | 0.81 | 0.23 |
| 75 | 11 | 6 | 0.16 | 0.57 | / | 0.37 | 0.07 | 0.08 | 0.87 | 0.22 |

| T/ | RH/ | Time/ | RRT | | | | | | |
|---|---|---|---|---|---|---|---|---|---|
| ° C. | % | day | 0.88 | 1.00 | 1.09 | 1.17 | 1.27 | 1.59 | 1.92 |
| 0 | 0 | 0 | 0.14 | 95.86 | / | 2.10 | 0.06 | / | / |
| 0 | 0 | 0 | 0.13 | 95.89 | / | 2.10 | 0.06 | / | / |
| 55 | 0 | 10 | 0.13 | 95.41 | 0.20 | 2.29 | 0.06 | 0.11 | / |
| 55 | 0 | 21 | 0.14 | 94.80 | / | 2.38 | 0.06 | 0.54 | / |
| 55 | 11 | 7 | 0.14 | 95.75 | / | 2.11 | 0.04 | 0.13 | / |
| 55 | 11 | 21 | 0.14 | 95.18 | 0.20 | 2.31 | 0.05 | 0.22 | / |
| 65 | 0 | 3 | 0.13 | 95.28 | 0.20 | 2.30 | 0.06 | 0.18 | / |
| 65 | 0 | 21 | 0.15 | 94.62 | 0.20 | 2.34 | 0.06 | 0.56 | / |
| 65 | 11 | 2 | 0.13 | 95.41 | 0.20 | 2.31 | 0.06 | 0.09 | / |
| 65 | 11 | 17 | 0.14 | 94.61 | 0.19 | 2.41 | 0.06 | 0.59 | / |
| 70 | 11 | 1 | 0.13 | 95.41 | 0.20 | 2.31 | 0.06 | 0.09 | / |
| 70 | 11 | 10 | 0.14 | 94.45 | 0.18 | 2.37 | 0.04 | 0.68 | / |
| 75 | 0 | 1 | 0.13 | 95.39 | 0.19 | 2.28 | 0.06 | 0.12 | / |
| 75 | 0 | 7 | 0.15 | 94.07 | 0.17 | 2.36 | 0.05 | 1.13 | / |
| 75 | 11 | 1 | 0.13 | 95.32 | 0.19 | 2.32 | 0.06 | 0.09 | / |
| 75 | 11 | 6 | 0.14 | 93.93 | 0.20 | 2.39 | 0.05 | 0.95 | / |

TABLE 3
Test result of related substance of composition A3

| T/ | RH/ | Time/ | RRT | | | | | | |
|---|---|---|---|---|---|---|---|---|---|
| ° C. | % | day | 0.26 | 0.31 | 0.57 | 0.58 | 0.63 | 0.75 | 0.78 |
| 0 | 0 | 0 | 0.53 | 0.35 | / | / | 0.09 | 0.72 | 0.24 |
| 0 | 0 | 0 | 0.52 | 0.34 | / | / | 0.09 | 0.74 | 0.24 |
| 55 | 0 | 10 | 0.57 | 0.34 | / | 0.06 | 0.07 | 0.74 | 0.24 |
| 55 | 0 | 21 | 0.62 | 0.37 | / | 0.07 | 0.10 | 0.74 | 0.24 |
| 55 | 11 | 7 | 0.60 | 0.34 | / | / | 0.10 | 0.73 | 0.24 |
| 55 | 11 | 21 | 0.62 | 0.35 | / | / | 0.10 | 0.74 | 0.25 |
| 65 | 0 | 3 | 0.56 | 0.34 | 0.02 | 0.05 | 0.10 | 0.74 | 0.25 |
| 65 | 0 | 21 | 0.67 | 0.37 | 0.07 | 0.16 | 0.08 | 0.76 | 0.24 |
| 65 | 11 | 2 | 0.54 | 0.34 | / | / | 0.07 | 0.74 | 0.24 |
| 65 | 11 | 17 | 0.86 | 0.37 | 0.04 | 0.10 | 0.07 | 0.75 | 0.25 |
| 70 | 11 | 1 | 0.54 | 0.34 | / | / | 0.07 | 0.74 | 0.24 |
| 70 | 11 | 10 | 0.55 | 0.33 | 0.07 | 0.16 | 0.08 | 0.75 | 0.26 |
| 75 | 0 | 1 | 0.56 | 0.34 | 0.02 | 0.05 | 0.08 | 0.74 | 0.24 |
| 75 | 0 | 7 | 0.71 | 0.39 | 0.07 | 0.17 | 0.08 | 0.78 | 0.25 |
| 75 | 11 | 1 | 0.65 | 0.34 | / | 0.07 | 0.06 | 0.76 | 0.24 |
| 75 | 11 | 6 | 0.58 | 0.32 | 0.10 | 0.25 | 0.08 | 0.77 | 0.26 |
| T/ | RH/ | Time/ | RRT | | | | | | |
| ° C. | % | day | 0.88 | 0.96 | 1.00 | 1.10 | 1.17 | 1.27 | 1.59 |
| 0 | 0 | 0 | 0.14 | / | 95.67 | / | 2.19 | 0.06 | / |
| 0 | 0 | 0 | 0.14 | / | 95.67 | / | 2.20 | 0.06 | / |
| 55 | 0 | 10 | 0.14 | 0.75 | 94.83 | / | 2.18 | 0.06 | / |
| 55 | 0 | 21 | 0.14 | 1.10 | 94.30 | 0.10 | 2.17 | 0.06 | / |
| 55 | 11 | 7 | 0.14 | / | 95.59 | / | 2.20 | 0.06 | / |
| 55 | 11 | 21 | 0.14 | 0.80 | 94.76 | / | 2.19 | 0.06 | / |
| 65 | 0 | 3 | 0.14 | 0.80 | 94.77 | / | 2.17 | 0.06 | / |
| 65 | 0 | 21 | 0.14 | 2.24 | 93.00 | / | 2.16 | 0.06 | 0.06 |
| 65 | 11 | 2 | 0.14 | 0.53 | 95.13 | / | 2.20 | 0.06 | / |
| 65 | 11 | 17 | 0.14 | 2.05 | 93.11 | / | 2.20 | 0.06 | / |
| 70 | 11 | 1 | 0.14 | 0.58 | 94.98 | 0.10 | 2.20 | 0.06 | / |
| 70 | 11 | 10 | 0.14 | 2.90 | 92.57 | / | 2.19 | / | / |
| 75 | 0 | 1 | 0.14 | 1.00 | 94.58 | / | 2.17 | 0.06 | / |
| 75 | 0 | 7 | 0.14 | 3.22 | 93.18 | / | 2.19 | 0.06 | 0.11 |
| 75 | 11 | 1 | 0.14 | 1.04 | 94.43 | / | 2.20 | 0.06 | / |
| 75 | 11 | 6 | 0.14 | 3.61 | 91.61 | / | 2.17 | 0.06 | 0.05 |

TABLE 4
Test result of related substance of composition A5

| T/ | RH/ | Time/ | RRT | | | | | | |
|---|---|---|---|---|---|---|---|---|---|
| ° C. | % | day | 0.25 | 0.26 | 0.30 | 0.37 | 0.57 | 0.61 | 0.74 |
| 0 | 0 | 0 | 0.07 | 0.31 | 0.34 | / | / | 0.08 | 0.72 |
| 0 | 0 | 0 | 0.07 | 0.31 | 0.33 | / | / | 0.08 | 0.72 |
| 55 | 0 | 10 | 0.06 | 0.32 | 0.34 | / | 0.04 | 0.08 | 0.73 |
| 55 | 0 | 21 | 0.10 | 0.37 | 0.35 | / | 0.08 | 0.09 | 0.73 |
| 55 | 11 | 7 | 0.06 | 0.31 | 0.33 | / | / | 0.08 | 0.74 |
| 55 | 11 | 21 | 0.09 | 0.35 | 0.37 | / | 0.05 | 0.08 | 0.73 |
| 65 | 0 | 3 | 0.07 | 0.31 | 0.34 | / | 0.04 | 0.08 | 0.74 |
| 65 | 0 | 21 | / | 0.43 | 0.35 | / | 0.18 | 0.10 | 0.74 |
| 65 | 11 | 2 | 0.06 | 0.31 | 0.34 | / | / | 0.08 | 0.72 |
| 65 | 11 | 17 | 0.09 | 0.37 | 0.33 | / | 0.31 | 0.11 | 0.73 |
| 70 | 11 | 1 | 0.06 | 0.30 | 0.32 | / | 0.06 | 0.08 | 0.73 |
| 70 | 11 | 10 | / | 0.46 | 0.27 | / | 0.53 | 0.11 | 0.75 |
| 75 | 0 | 1 | 0.07 | 0.33 | 0.34 | / | 0.04 | 0.07 | 0.73 |
| 75 | 0 | 7 | / | 0.46 | 0.36 | / | 0.23 | 0.11 | 0.76 |
| 75 | 11 | 1 | 0.08 | 0.32 | 0.32 | / | 0.14 | 0.11 | 0.74 |
| 75 | 11 | 6 | / | 0.55 | 0.27 | 0.05 | 0.62 | 0.17 | 0.75 |
| T/ | RH/ | Time/ | RRT | | | | | | |
| ° C. | % | day | 0.78 | 0.88 | 0.95 | 1.00 | 1.16 | 1.26 | 1.58 |
| 0 | 0 | 0 | 0.25 | 0.13 | / | 95.87 | 2.17 | 0.06 | / |
| 0 | 0 | 0 | 0.25 | 0.13 | / | 95.90 | 2.16 | 0.06 | / |
| 55 | 0 | 10 | 0.24 | 0.14 | 0.54 | 95.30 | 2.16 | 0.06 | / |
| 55 | 0 | 21 | 0.24 | 0.14 | 0.74 | 94.96 | 2.16 | 0.06 | / |
| 55 | 11 | 7 | 0.24 | 0.13 | / | 96.22 | 2.19 | 0.06 | / |
| 55 | 11 | 21 | 0.23 | 0.13 | 0.60 | 95.11 | 2.18 | 0.06 | / |
| 65 | 0 | 3 | 0.23 | 0.13 | 0.54 | 95.28 | 2.18 | 0.06 | / |
| 65 | 0 | 21 | 0.24 | 0.14 | 1.57 | 93.98 | 2.18 | 0.08 | / |
| 65 | 11 | 2 | 0.24 | 0.13 | 0.49 | 95.37 | 2.19 | 0.06 | / |
| 65 | 11 | 17 | 0.24 | 0.14 | 2.44 | 92.98 | 2.20 | 0.06 | / |
| 70 | 11 | 1 | 0.24 | 0.14 | 0.78 | 95.04 | 2.18 | 0.06 | / |
| 70 | 11 | 10 | 0.23 | 0.15 | 5.26 | 89.73 | 2.37 | 0.07 | 0.08 |
| 75 | 0 | 1 | 0.24 | 0.13 | 0.69 | 95.13 | 2.17 | 0.06 | / |
| 75 | 0 | 7 | 0.24 | 0.14 | 2.16 | 93.97 | 2.21 | 0.08 | 0.11 |
| 75 | 11 | 1 | 0.24 | 0.14 | 2.15 | 93.55 | 2.16 | 0.06 | / |
| 75 | 11 | 6 | 0.21 | 0.15 | 10.19 | 84.65 | 2.33 | 0.07 | / |

TABLE 5
Test result of related substance of composition A6

| T/ | RH/ | Time/ | RRT | | | | | | |
|---|---|---|---|---|---|---|---|---|---|
| ° C. | % | day | 0.25 | 0.26 | 0.29 | 0.30 | 0.32 | 0.36 | 0.40 |
| 0 | 0 | 0 | 0.14 | 0.38 | / | 0.37 | / | / | / |
| 0 | 0 | 0 | 0.55 | 0.58 | / | 0.47 | / | / | / |
| 55 | 0 | 10 | / | 0.77 | 0.15 | 0.40 | / | 0.47 | / |
| 55 | 0 | 21 | / | 3.93 | 2.46 | 3.35 | 0.61 | 4.15 | / |
| 55 | 11 | 7 | 0.48 | 0.72 | 0.32 | 0.53 | 0.06 | 1.32 | / |
| 55 | 11 | 21 | / | 3.23 | / | 2.02 | 0.42 | 2.69 | / |
| 65 | 0 | 3 | / | 1.81 | 0.74 | 0.92 | 0.11 | 1.96 | / |
| 65 | 0 | 21 | / | 10.94 | 5.65 | 3.15 | 2.82 | 6.47 | 0.18 |
| 65 | 11 | 2 | / | 1.91 | 0.58 | 0.83 | 0.12 | 2.21 | / |
| 65 | 11 | 17 | / | 17.50 | 6.54 | 3.41 | 2.12 | 12.13 | / |
| 70 | 11 | 1 | / | 2.70 | 0.90 | 1.34 | 0.25 | 3.03 | / |
| 70 | 11 | 10 | / | 20.03 | 7.32 | 3.79 | 2.41 | 13.27 | / |
| 75 | 0 | 1 | / | 3.54 | 2.39 | 2.47 | 0.40 | 4.49 | / |
| 75 | 0 | 7 | / | 34.31 | 7.62 | 4.72 | 2.50 | 15.25 | / |
| 75 | 11 | 1 | / | 4.86 | 1.97 | 2.88 | 0.58 | 5.35 | / |
| 75 | 11 | 6 | / | 30.41 | 11.01 | 6.06 | 3.05 | 17.35 | / |

| T/ | RH/ | Time/ | RRT | | | | | | |
|---|---|---|---|---|---|---|---|---|---|
| ° C. | % | day | 0.43 | 0.54 | 0.58 | 0.61 | 0.64 | 0.74 | 0.77 |
| 0 | 0 | 0 | / | / | / | 0.08 | / | 0.39 | 0.24 |
| 0 | 0 | 0 | / | / | / | 0.08 | / | 0.70 | 0.23 |
| 55 | 0 | 10 | / | / | / | 0.08 | / | 0.71 | 0.23 |
| 55 | 0 | 21 | / | 0.11 | 0.14 | 0.29 | 1.09 | 0.79 | 0.31 |
| 55 | 11 | 7 | / | 0.06 | 0.17 | 0.20 | 0.46 | 0.72 | 0.22 |
| 55 | 11 | 21 | 0.06 | 0.05 | 0.05 | 0.46 | 2.02 | 1.23 | 0.62 |
| 65 | 0 | 3 | / | 0.06 | 0.15 | 0.23 | 0.91 | 0.77 | 0.25 |
| 65 | 0 | 21 | 0.08 | 0.23 | 0.20 | 0.24 | 0.59 | 0.64 | 0.22 |
| 65 | 11 | 2 | / | 0.06 | 0.16 | 0.26 | 1.00 | 0.73 | 0.23 |
| 65 | 11 | 17 | 0.11 | 0.11 | / | 0.36 | 1.88 | 1.16 | / |
| 70 | 11 | 1 | / | 0.08 | 0.15 | 0.30 | 1.39 | 1.03 | / |
| 70 | 11 | 10 | 0.14 | 0.17 | / | 0.29 | 1.63 | 0.95 | / |
| 75 | 0 | 1 | / | 0.10 | 0.11 | 0.26 | 1.23 | 1.09 | / |
| 75 | 0 | 7 | 0.06 | 0.33 | / | 0.20 | 1.25 | 1.04 | / |
| 75 | 11 | 1 | / | 0.10 | 0.12 | 0.35 | 2.01 | 1.53 | / |
| 75 | 11 | 6 | 0.16 | 0.19 | / | 0.30 | 2.07 | 1.37 | / |
| T/ | RH/ | Time/ | RRT | | | | | | |
| ° C. | % | day | 0.81 | 0.88 | 0.98 | 1.00 | 1.09 | 1.13 | 1.26 |
| 0 | 0 | 0 | / | 0.12 | / | 95.79 | / | 2.12 | 0.06 |
| 0 | 0 | 0 | / | 0.13 | / | 95.05 | / | 2.15 | 0.06 |
| 55 | 0 | 10 | / | 0.13 | / | 94.55 | 0.21 | 2.26 | 0.06 |
| 55 | 0 | 21 | 0.09 | 0.15 | / | 80.72 | / | 1.72 | 0.09 |
| 55 | 11 | 7 | / | 0.14 | / | 92.44 | / | 2.09 | 0.06 |
| 55 | 11 | 21 | 1.17 | 0.50 | 1.61 | 81.53 | 2.11 | / | 0.09 |
| 65 | 0 | 3 | / | 0.12 | / | 89.42 | / | 2.34 | 0.22 |
| 65 | 0 | 21 | / | 0.12 | / | 64.63 | 1.31 | 1.53 | / |
| 65 | 11 | 2 | / | 0.13 | / | 89.53 | / | 2.12 | 0.10 |
| 65 | 11 | 17 | 0.44 | 0.60 | / | 52.32 | / | 1.07 | / |
| 70 | 11 | 1 | / | 0.12 | / | 86.73 | / | 1.87 | 0.11 |
| 70 | 11 | 10 | 0.39 | 0.63 | 28.12 | 19.33 | / | 0.99 | / |
| 75 | 0 | 1 | / | 0.12 | / | 81.86 | / | 1.75 | 0.09 |
| 75 | 0 | 7 | 0.08 | / | 15.22 | 13.84 | 1.05 | 0.41 | 0.18 |
| 75 | 11 | 1 | 0.09 | 0.17 | / | 78.23 | / | 1.63 | 0.14 |
| 75 | 11 | 6 | 0.29 | / | 8.08 | 18.06 | / | 0.38 | / |
| T/ | RH/ | Time/ | RRT | | | | | | |
| ° C. | % | day | 1.45 | 1.53 | 1.75 | 1.84 | 1.93 | 2.02 | 2.07 |
| 0 | 0 | 0 | / | / | / | / | / | / | / |
| 0 | 0 | 0 | / | / | / | / | / | / | / |
| 55 | 0 | 10 | / | / | / | / | / | / | / |
| 55 | 0 | 21 | / | / | / | / | / | / | / |
| 55 | 11 | 7 | / | / | / | / | / | / | / |
| 55 | 11 | 21 | / | / | 0.13 | / | / | / | / |
| 65 | 0 | 3 | / | / | / | / | / | / | / |
| 65 | 0 | 21 | 0.13 | 0.43 | / | / | / | 0.43 | / |
| 65 | 11 | 2 | / | / | / | / | / | / | / |
| 65 | 11 | 17 | 0.13 | 0.12 | / | / | / | / | / |
| 70 | 11 | 1 | / | / | / | / | / | / | / |
| 70 | 11 | 10 | 0.10 | 0.18 | / | / | / | / | 0.26 |
| 75 | 0 | 1 | / | 0.11 | / | / | / | / | / |
| 75 | 0 | 7 | 0.19 | 0.49 | 0.23 | 0.12 | / | 0.92 | / |
| 75 | 11 | 1 | / | / | / | / | / | / | / |
| 75 | 11 | 6 | 1.45 | 1.53 | 1.75 | 1.84 | 1.93 | 2.02 | 2.07 |

TABLE 6
Test result of related substance of composition A7

| T/ | RH/ | Time/ | RRT | | | | | |
|---|---|---|---|---|---|---|---|---|
| ° C. | % | day | 0.26 | 0.31 | 0.57 | 0.62 | 0.75 | 0.78 |
| 0 | 0 | 0 | 0.32 | 0.33 | / | 0.08 | 0.72 | 0.24 |
| 0 | 0 | 0 | 0.32 | 0.33 | / | 0.08 | 0.73 | 0.24 |
| 55 | 0 | 10 | 0.33 | 0.33 | 0.08 | 0.11 | 0.72 | 0.23 |
| 55 | 0 | 21 | 0.39 | 0.33 | 0.13 | 0.13 | 0.72 | 0.24 |
| 55 | 11 | 7 | 0.33 | 0.33 | / | 0.10 | 0.74 | 0.23 |
| 55 | 11 | 21 | 0.32 | 0.31 | 0.06 | 0.10 | 0.71 | 0.25 |
| 65 | 0 | 3 | 0.33 | 0.33 | 0.07 | 0.11 | 0.73 | 0.24 |
| 65 | 0 | 21 | 0.38 | 0.34 | 0.25 | 0.18 | 0.73 | 0.24 |
| 65 | 11 | 2 | 0.34 | 0.33 | / | 0.08 | 0.73 | 0.24 |
| 65 | 11 | 17 | 0.37 | 0.34 | 0.14 | 0.14 | 0.74 | 0.24 |
| 70 | 11 | 1 | 0.32 | 0.33 | 0.04 | 0.10 | 0.73 | 0.24 |
| 70 | 11 | 10 | 0.46 | 0.36 | 0.18 | 0.16 | 0.73 | 0.24 |
| 75 | 0 | 1 | 0.33 | 0.33 | 0.07 | 0.11 | 0.73 | 0.24 |
| 75 | 0 | 7 | 0.37 | 0.35 | 0.30 | 0.22 | 0.76 | 0.24 |
| 75 | 11 | 1 | 0.31 | 0.31 | 0.07 | 0.11 | 0.74 | 0.24 |
| 75 | 11 | 6 | 0.42 | 0.35 | 0.21 | 0.17 | 0.73 | 0.24 |

| T/ | RH/ | Time/ | RRT | | | | |
|---|---|---|---|---|---|---|---|
| ° C. | % | day | 0.88 | 1.00 | 1.16 | 1.26 | 1.58 |
| 0 | 0 | 0 | 0.13 | 95.94 | 2.17 | 0.06 | / |
| 0 | 0 | 0 | 0.13 | 95.94 | 2.17 | 0.06 | / |
| 55 | 0 | 10 | 0.13 | 95.82 | 2.18 | 0.06 | / |
| 55 | 0 | 21 | 0.14 | 95.60 | 2.19 | 0.06 | 0.09 |
| 55 | 11 | 7 | 0.13 | 95.88 | 2.19 | 0.06 | / |
| 55 | 11 | 21 | 0.14 | 95.87 | 2.19 | 0.06 | / |
| 65 | 0 | 3 | 0.14 | 95.82 | 2.18 | 0.06 | / |
| 65 | 0 | 21 | 0.14 | 95.40 | 2.20 | 0.06 | / |
| 65 | 11 | 2 | 0.14 | 95.90 | 2.19 | 0.06 | / |
| 65 | 11 | 17 | 0.14 | 95.52 | 2.23 | 0.06 | 0.09 |
| 70 | 11 | 1 | 0.13 | 95.86 | 2.18 | 0.06 | / |
| 70 | 11 | 10 | 0.14 | 95.35 | 2.22 | 0.06 | 0.08 |
| 75 | 0 | 1 | 0.14 | 95.82 | 2.18 | 0.06 | / |
| 75 | 0 | 7 | 0.14 | 95.25 | 2.22 | 0.06 | 0.10 |
| 75 | 11 | 1 | 0.14 | 95.83 | 2.19 | 0.06 | / |
| 75 | 11 | 6 | 0.14 | 95.36 | 2.21 | 0.06 | 0.10 |

TABLE 7
Test result of related substance of composition A9

| T/ | RH/ | Time/ | RRT | | | | | | |
|---|---|---|---|---|---|---|---|---|---|
| ° C. | % | day | 0.25 | 0.26 | 0.30 | 0.38 | 0.40 | 0.59 | 0.62 |
| 0 | 0 | 0 | / | 0.38 | 0.33 | / | / | / | 0.08 |
| 0 | 0 | 0 | / | 0.39 | 0.32 | / | / | / | 0.08 |
| 55 | 0 | 10 | / | 0.46 | 0.48 | / | / | / | 0.09 |
| 55 | 0 | 21 | / | 1.12 | 0.35 | 0.09 | 0.08 | / | 0.10 |
| 55 | 11 | 7 | / | 0.48 | 0.32 | / | / | / | 0.08 |
| 55 | 11 | 21 | / | 0.62 | 0.32 | / | / | / | 0.10 |
| 65 | 0 | 3 | / | 0.47 | 0.34 | / | / | / | 0.10 |
| 65 | 0 | 21 | 0.35 | 0.53 | 0.37 | 0.05 | 0.05 | 0.04 | 0.09 |
| 65 | 11 | 2 | / | 0.43 | 0.39 | / | / | / | 0.09 |
| 65 | 11 | 17 | / | 1.03 | 0.36 | / | / | / | / |
| 70 | 11 | 1 | / | 0.49 | 0.33 | / | / | / | 0.09 |
| 70 | 11 | 10 | / | 0.74 | 0.56 | / | / | 0.07 | 0.11 |
| 75 | 0 | 1 | / | 0.40 | 0.33 | / | / | / | 0.11 |
| 75 | 0 | 7 | / | 0.77 | 0.44 | / | / | 0.06 | 0.13 |
| 75 | 11 | 1 | / | 0.45 | 0.31 | / | / | / | 0.10 |
| 75 | 11 | 6 | / | 0.76 | 0.60 | / | / | 0.07 | 0.11 |
| T/ | RH/ | Time/ | RRT | | | | | | |
| ° C. | % | day | 0.74 | 0.78 | 0.88 | 1.00 | 1.11 | 1.16 | 1.27 |
| 0 | 0 | 0 | 0.75 | 0.24 | 0.14 | 95.81 | / | 2.21 | 0.06 |
| 0 | 0 | 0 | 0.76 | 0.23 | 0.14 | 95.81 | / | 2.21 | 0.06 |
| 55 | 0 | 10 | 1.66 | 0.21 | 0.16 | 93.36 | / | 2.68 | / |
| 55 | 0 | 21 | 2.48 | / | 0.19 | 88.66 | / | 4.35 | / |
| 55 | 11 | 7 | 1.30 | / | 0.17 | 94.92 | / | 2.82 | 0.10 |
| 55 | 11 | 21 | 1.56 | 0.21 | 0.17 | 93.47 | / | 2.73 | / |
| 65 | 0 | 3 | 1.87 | 0.20 | 0.16 | 93.51 | / | 2.67 | 0.05 |
| 65 | 0 | 21 | 3.48 | / | 0.23 | 88.95 | 0.09 | 3.52 | / |
| 65 | 11 | 2 | 1.54 | / | 0.16 | 94.22 | / | 2.89 | 0.12 |
| 65 | 11 | 17 | 4.62 | / | 0.19 | 86.84 | 0.54 | 4.19 | 0.08 |
| 70 | 11 | 1 | 1.44 | 0.22 | 0.16 | 94.49 | / | 2.53 | / |
| 70 | 11 | 10 | 4.76 | / | 0.23 | 84.42 | / | 5.20 | / |
| 75 | 0 | 1 | 1.83 | 0.21 | 0.16 | 93.76 | / | 2.70 | / |
| 75 | 0 | 7 | 6.03 | / | 0.24 | 84.71 | 0.79 | 4.81 | / |
| 75 | 11 | 1 | 2.10 | / | 0.18 | 93.21 | / | 3.13 | 0.11 |
| 75 | 11 | 6 | 5.32 | / | 0.24 | 83.36 | / | 5.42 | / |

| T/ | RH/ | Time/ | RRT | | | | | |
|---|---|---|---|---|---|---|---|---|
| ° C. | % | day | 1.31 | 1.42 | 1.58 | 1.89 | 2.11 | 2.47 |
| 0 | 0 | 0 | / | / | / | / | / | / |
| 0 | 0 | 0 | / | / | / | / | / | / |
| 55 | 0 | 10 | / | 0.23 | 0.43 | / | 0.22 | / |
| 55 | 0 | 21 | / | 0.79 | 1.13 | 0.10 | 0.57 | / |
| 55 | 11 | 7 | / | 0.12 | 0.12 | 0.06 | / | / |
| 55 | 11 | 21 | / | 0.22 | 0.40 | 0.20 | / | / |
| 65 | 0 | 3 | / | 0.29 | 0.22 | 0.12 | / | / |
| 65 | 0 | 21 | / | 0.24 | 1.47 | 0.53 | / | / |
| 65 | 11 | 2 | / | 0.16 | / | / | / | / |
| 65 | 11 | 17 | 0.05 | 0.27 | 1.14 | 0.67 | / | / |
| 70 | 11 | 1 | / | 0.18 | 0.07 | / | / | / |
| 70 | 11 | 10 | / | 0.46 | 1.41 | 0.60 | 1.44 | / |
| 75 | 0 | 1 | / | 0.27 | 0.24 | / | / | / |
| 75 | 0 | 7 | / | 0.38 | 1.46 | 0.17 | / | / |
| 75 | 11 | 1 | / | 0.26 | 0.15 | / | / | / |
| 75 | 11 | 6 | / | 0.40 | 1.77 | 0.77 | 1.10 | 0.08 |

TABLE 8
Test result of related substance of composition A10

| T/ | RH/ | Time/ | RRT | | | | | | | |
|---|---|---|---|---|---|---|---|---|---|---|
| ° C. | % | day | 0.25 | 0.27 | 0.31 | 0.35 | 0.36 | 0.47 | 0.49 | 0.63 |
| 0 | 0 | 0 | / | 0.62 | 0.68 | 0.41 | 0.68 | / | 0.82 | / |
| 0 | 0 | 0 | / | 0.50 | 0.65 | / | 1.12 | / | 0.79 | 0.12 |
| 55 | 0 | 10 | / | 0.68 | 0.71 | 0.43 | 0.72 | / | 0.90 | / |
| 55 | 0 | 21 | / | 0.71 | 0.71 | 0.42 | 0.72 | / | 0.91 | / |
| 55 | 11 | 7 | / | 0.69 | 0.66 | 0.38 | 0.70 | 0.14 | 0.70 | / |
| 55 | 11 | 21 | / | 0.69 | 0.73 | 0.44 | 0.69 | / | 0.95 | / |
| 65 | 0 | 3 | / | 0.66 | 0.70 | 0.42 | 0.71 | / | 0.88 | / |
| 65 | 0 | 21 | / | 0.76 | 0.69 | 0.41 | 0.70 | / | 0.88 | / |
| 65 | 11 | 2 | / | 0.69 | 0.66 | 0.40 | 0.69 | 0.21 | 0.65 | / |
| 65 | 11 | 17 | / | 0.77 | 0.68 | 0.41 | 0.71 | 0.19 | 0.78 | / |
| 70 | 11 | 1 | / | 0.68 | 0.70 | 0.45 | 0.69 | / | 0.90 | / |
| 70 | 11 | 10 | 0.06 | 0.66 | 0.71 | 0.44 | 0.68 | / | 0.90 | / |
| 75 | 0 | 1 | / | 0.71 | 0.71 | 0.45 | 0.66 | 0.18 | 0.76 | / |
| 75 | 0 | 7 | / | 0.66 | 0.68 | 0.43 | 0.70 | / | 0.88 | / |
| 75 | 11 | 1 | / | 0.73 | 0.68 | / | 1.08 | / | 0.92 | / |
| 75 | 11 | 6 | / | 0.74 | 0.72 | 0.43 | 0.69 | 0.21 | 0.79 | / |

| T/ | RH/ | Time/ | RRT | | | | | | |
|---|---|---|---|---|---|---|---|---|---|
| ° C. | % | day | 0.76 | 0.81 | 0.89 | 1.00 | 1.19 | 1.27 | 1.60 |
| 0 | 0 | 0 | 0.59 | 0.25 | 0.14 | 92.81 | 2.75 | 0.25 | / |
| 0 | 0 | 0 | 0.59 | 0.17 | / | 93.08 | 2.73 | 0.24 | / |
| 55 | 0 | 10 | 0.64 | 0.21 | 0.18 | 92.90 | 3.08 | 0.22 | / |
| 55 | 0 | 21 | 0.63 | 0.18 | 0.16 | 92.11 | 3.23 | 0.21 | / |
| 55 | 11 | 7 | 0.50 | 0.15 | / | 92.68 | 2.98 | 0.21 | 0.19 |
| 55 | 11 | 21 | 0.64 | 0.24 | / | 91.85 | 3.06 | 0.20 | 0.51 |
| 65 | 0 | 3 | 0.65 | 0.23 | 0.16 | 92.33 | 3.06 | 0.21 | / |
| 65 | 0 | 21 | 0.55 | 0.16 | / | 92.30 | 3.35 | 0.20 | / |
| 65 | 11 | 2 | 0.55 | 0.16 | / | 92.76 | 3.02 | 0.20 | / |
| 65 | 11 | 17 | 0.68 | 0.18 | / | 91.16 | 3.30 | 0.19 | 0.96 |
| 70 | 11 | 1 | 0.67 | 0.22 | 0.14 | 92.15 | 3.18 | 0.20 | / |
| 70 | 11 | 10 | 0.61 | 0.18 | / | 92.40 | 2.95 | 0.22 | 0.20 |
| 75 | 0 | 1 | 0.68 | 0.19 | / | 91.15 | 3.20 | 0.20 | 1.11 |
| 75 | 0 | 7 | 0.64 | 0.18 | 0.14 | 92.36 | 3.11 | 0.21 | / |
| 75 | 11 | 1 | 0.67 | 0.19 | / | 91.61 | 3.07 | 0.19 | 0.86 |
| 75 | 11 | 6 | 0.68 | 0.19 | / | 90.49 | 3.42 | 0.19 | 1.44 |

TABLE 9
Test result of related substance of composition A11

| T/ | RH/ | Time/ | RRT | | | | | | |
|---|---|---|---|---|---|---|---|---|---|
| ° C. | % | day | 0.15 | 0.25 | 0.30 | 0.31 | 0.43 | 0.62 | 0.75 |
| 0 | 0 | 0 | / | 22.16 | / | 0.23 | / | 0.06 | 0.52 |
| 0 | 0 | 0 | 0.22 | 22.03 | / | 0.36 | / | 0.07 | 0.54 |
| 55 | 0 | 10 | / | 23.53 | / | 0.22 | / | 0.55 | 0.71 |
| 55 | 0 | 21 | / | 22.41 | 0.09 | 0.22 | / | 0.08 | 0.56 |
| 55 | 11 | 7 | / | 22.73 | 0.09 | 0.19 | / | 0.16 | 0.72 |
| 55 | 11 | 21 | / | 22.48 | 0.07 | 0.23 | / | 0.10 | 0.77 |
| 65 | 0 | 3 | / | 25.69 | 0.40 | 0.24 | 0.08 | 1.07 | 0.76 |
| 65 | 0 | 21 | / | 22.51 | 0.06 | 0.22 | / | 0.08 | 0.59 |
| 65 | 11 | 2 | 0.43 | 24.38 | 0.27 | 0.35 | / | 0.68 | 0.69 |
| 65 | 11 | 17 | / | 22.35 | 0.09 | 0.23 | / | 0.08 | 0.58 |
| 70 | 11 | 1 | / | 25.02 | 0.23 | 0.22 | / | 0.67 | 0.68 |
| 70 | 11 | 10 | / | 37.66 | 0.44 | 0.33 | 0.12 | 2.92 | 1.11 |
| 75 | 0 | 1 | 0.58 | 22.36 | 0.15 | 0.30 | / | 0.08 | 0.58 |
| 75 | 0 | 7 | / | 24.93 | 0.19 | 0.22 | 0.06 | 0.78 | 0.79 |
| 75 | 11 | 1 | 0.15 | 0.25 | 0.30 | 0.31 | 0.43 | 0.62 | 0.75 |
| 75 | 11 | 6 | / | 22.16 | / | 0.23 | / | 0.06 | 0.52 |
| T/ | RH/ | Time/ | RRT | | | | | | |
| ° C. | % | day | 0.78 | 0.83 | 0.88 | 0.93 | 1.00 | 1.05 | 1.13 |
| 0 | 0 | 0 | 0.17 | / | 0.10 | / | 74.61 | / | / |
| 0 | 0 | 0 | 0.17 | / | 0.10 | / | 74.48 | / | / |
| 55 | 0 | 10 | / | / | 0.16 | 0.07 | 70.73 | / | / |
| 55 | 0 | 21 | 0.17 | / | 0.10 | / | 74.25 | / | 2.11 |
| 55 | 11 | 7 | / | / | 0.14 | 0.10 | 72.60 | / | / |
| 55 | 11 | 21 | / | / | 0.13 | 0.05 | 73.91 | / | / |
| 65 | 0 | 3 | / | 0.08 | 0.22 | 0.17 | 61.54 | 3.47 | / |
| 65 | 0 | 21 | 0.17 | / | 0.10 | / | 74.16 | / | 2.10 |
| 65 | 11 | 2 | 0.14 | 0.07 | 0.16 | 0.25 | 67.18 | / | / |
| 65 | 11 | 17 | 0.17 | / | 0.10 | / | 74.31 | / | / |
| 70 | 11 | 1 | 0.15 | 0.08 | 0.14 | 0.22 | 65.80 | / | / |
| 70 | 11 | 10 | / | 0.15 | / | / | 77.32 | 10.91 | 1.99 |
| 75 | 0 | 1 | 0.18 | / | 0.13 | 0.06 | 73.46 | / | / |
| 75 | 0 | 7 | / | / | 0.14 | 0.22 | 63.80 | 2.24 | / |
| 75 | 11 | 1 | 0.78 | 0.83 | 0.88 | 0.93 | 1.00 | 1.05 | 1.13 |
| 75 | 11 | 6 | 0.17 | / | 0.10 | / | 74.61 | / | / |

| T/ | RH/ | Time/ | RRT | | | | | | | |
|---|---|---|---|---|---|---|---|---|---|---|
| ° C. | % | day | 1.15 | 1.27 | 1.45 | 1.52 | 1.57 | 1.90 | 2.11 | 2.46 |
| 0 | 0 | 0 | 2.09 | 0.05 | / | / | / | / | / | / |
| 0 | 0 | 0 | 2.04 | / | / | / | / | / | / | / |
| 55 | 0 | 10 | 2.58 | 0.12 | / | 0.14 | 0.61 | 0.18 | 0.41 | / |
| 55 | 0 | 21 | / | / | / | / | / | / | / | / |
| 55 | 11 | 7 | 2.20 | 0.07 | 0.12 | / | 0.24 | 0.19 | 0.44 | / |
| 55 | 11 | 21 | 2.12 | 0.08 | / | / | 0.06 | / | / | / |
| 65 | 0 | 3 | 2.27 | / | / | 0.34 | 1.10 | 0.65 | 1.93 | / |
| 65 | 0 | 21 | / | / | / | / | / | / | / | / |
| 65 | 11 | 2 | 2.32 | / | / | 0.09 | 0.93 | 0.70 | 1.37 | / |
| 65 | 11 | 17 | 2.08 | / | / | / | / | / | / | / |
| 70 | 11 | 1 | 2.54 | / | 0.06 | 0.09 | 1.21 | 0.76 | 2.14 | / |
| 70 | 11 | 10 | 3.57 | / | / | 0.27 | 2.24 | 0.97 | 2.53 | 0.20 |
| 75 | 0 | 1 | 2.13 | / | / | / | / | / | / | / |
| 75 | 0 | 7 | 2.26 | / | 0.10 | 0.09 | 1.45 | 0.85 | 1.88 | / |
| 75 | 11 | 1 | 1.15 | 1.27 | 1.45 | 1.52 | 1.57 | 1.90 | 2.11 | 2.46 |
| 75 | 11 | 6 | 2.09 | 0.05 | / | / | / | / | / | / |

TABLE 10
Test result of related substance of composition A12

| T/ | RH/ | Time/ | RRT | | | | | | |
|---|---|---|---|---|---|---|---|---|---|
| ° C. | % | day | 0.15 | 0.17 | 0.27 | 0.30 | 0.32 | 0.38 | 0.63 |
| 0 | 0 | 0 | / | 0.95 | 0.31 | / | 0.34 | / | 0.08 |
| 0 | 0 | 0 | 0.73 | 1.40 | 0.49 | / | 0.71 | / | 0.07 |
| 55 | 0 | 10 | / | 1.33 | 0.39 | 0.23 | 0.36 | / | 0.08 |
| 55 | 0 | 21 | 1.38 | 2.20 | 0.60 | 0.45 | 0.57 | / | 0.07 |
| 55 | 11 | 7 | / | 1.16 | 0.39 | 0.30 | 0.35 | / | 0.08 |
| 55 | 11 | 21 | 1.74 | 2.24 | 0.62 | 1.58 | / | / | 0.07 |
| 65 | 0 | 3 | / | 1.31 | 0.36 | 0.14 | 0.36 | / | 0.08 |
| 65 | 0 | 21 | / | 3.65 | 0.66 | 0.55 | 0.42 | / | 0.06 |
| 65 | 11 | 2 | / | 1.19 | 0.35 | 0.13 | 0.35 | / | 0.08 |
| 65 | 11 | 17 | 1.33 | 3.66 | 0.96 | 2.32 | / | / | 0.07 |
| 70 | 11 | 1 | / | 1.21 | 0.38 | 0.12 | 0.35 | / | 0.07 |
| 70 | 11 | 10 | / | 3.03 | 0.70 | 1.86 | / | / | 0.06 |
| 75 | 0 | 1 | / | 1.30 | 0.37 | 0.14 | 0.36 | / | 0.08 |
| 75 | 0 | 7 | / | 4.07 | 0.81 | 0.66 | 0.58 | 0.07 | / |
| 75 | 11 | 1 | 2.72 | 2.43 | 0.64 | 0.38 | 0.73 | / | 0.07 |
| 75 | 11 | 6 | / | 3.12 | 0.70 | 1.52 | / | / | / |

| T/ | RH/ | Time/ | RRT | | | | | | |
|---|---|---|---|---|---|---|---|---|---|
| ° C. | % | day | 0.75 | 0.78 | 0.88 | 1.00 | 1.16 | 1.45 | 1.57 |
| 0 | 0 | 0 | 0.73 | 0.21 | 0.13 | 94.84 | 2.36 | 0.06 | |
| 0 | 0 | 0 | 0.69 | 0.20 | 0.13 | 93.28 | 2.30 | / | / |
| 55 | 0 | 10 | 0.73 | 0.21 | 0.16 | 92.08 | 2.57 | / | 1.85 |
| 55 | 0 | 21 | 0.71 | 0.21 | 0.19 | 90.59 | 2.51 | / | 0.52 |
| 55 | 11 | 7 | 0.76 | 0.22 | 0.15 | 93.59 | 2.66 | / | / |
| 55 | 11 | 21 | 0.70 | 0.21 | 0.16 | 86.68 | 2.79 | / | 2.80 |
| 65 | 0 | 3 | 0.73 | 0.21 | 0.15 | 93.48 | 2.52 | / | 0.65 |
| 65 | 0 | 21 | 0.77 | 0.23 | 0.42 | 76.87 | 3.42 | 0.55 | 10.07 |
| 65 | 11 | 2 | 0.75 | 0.21 | 0.18 | 94.08 | 2.55 | 0.06 | / |
| 65 | 11 | 17 | 0.84 | 0.20 | 0.32 | 79.64 | 3.17 | / | 0.18 |
| 70 | 11 | 1 | 0.76 | 0.21 | 0.14 | 94.07 | 2.58 | / | / |
| 70 | 11 | 10 | 0.87 | 0.22 | 0.33 | 79.28 | 3.74 | / | 0.34 |
| 75 | 0 | 1 | 0.74 | 0.22 | 0.14 | 93.36 | 2.57 | / | 0.73 |
| 75 | 0 | 7 | 0.83 | 0.22 | 0.54 | 73.31 | 3.95 | / | 0.83 |
| 75 | 11 | 1 | 0.75 | 0.19 | 0.14 | 89.21 | 2.54 | / | / |
| 75 | 11 | 6 | 0.87 | 0.23 | 0.43 | 76.90 | 3.31 | 0.49 | 10.18 |

| T/ | RH/ | Time/ | RRT | | | | | |
|---|---|---|---|---|---|---|---|---|
| ° C. | % | day | 1.71 | 1.83 | 1.89 | 2.09 | 2.17 | 2.45 |
| 0 | 0 | 0 | / | / | / | / | / | / |
| 0 | 0 | 0 | / | / | / | / | / | / |
| 55 | 0 | 10 | / | / | / | / | / | / |
| 55 | 0 | 21 | / | / | / | / | / | / |
| 55 | 11 | 7 | 0.34 | / | / | / | / | / |
| 55 | 11 | 21 | / | / | 0.25 | 0.16 | / | / |
| 65 | 0 | 3 | / | / | / | / | / | / |
| 65 | 0 | 21 | / | 0.13 | 0.67 | 0.88 | / | 0.64 |
| 65 | 11 | 2 | 0.08 | / | / | / | / | / |
| 65 | 11 | 17 | 5.83 | / | / | 0.60 | 0.73 | 0.16 |
| 70 | 11 | 1 | 0.10 | / | / | / | / | / |
| 70 | 11 | 10 | 7.57 | / | 0.09 | 0.76 | 1.15 | / |
| 75 | 0 | 1 | / | / | / | / | / | / |
| 75 | 0 | 7 | 11.33 | 0.07 | / | 0.33 | 1.37 | 1.01 |
| 75 | 11 | 1 | 0.18 | / | / | / | / | / |
| 75 | 11 | 6 | / | 0.09 | 0.82 | 0.87 | 0.23 | 0.24 |

TABLE 11
Test result of related substance of composition A13

| T/ | RH/ | Time/ | RRT | | | | | | | |
|---|---|---|---|---|---|---|---|---|---|---|
| ° C. | % | day | 0.12 | 0.27 | 0.30 | 0.34 | 0.39 | 0.55 | 0.63 | 0.66 |
| 0 | 0 | 0 | / | 0.79 | 25.39 | 0.31 | / | 0.06 | 0.12 | 0.05 |
| 0 | 0 | 0 | 1.30 | 0.82 | 23.98 | 0.31 | / | 0.07 | 0.12 | 0.05 |
| 55 | 0 | 10 | / | 0.72 | 23.99 | 0.26 | / | 0.06 | 0.12 | / |
| 55 | 0 | 21 | 1.14 | 0.67 | 20.48 | 0.31 | / | / | / | / |
| 55 | 11 | 7 | / | 0.75 | 23.84 | 0.31 | / | / | / | / |
| 55 | 11 | 21 | 1.59 | 0.92 | 19.74 | 0.30 | / | / | 0.13 | 0.05 |
| 65 | 0 | 3 | / | 0.72 | 24.10 | 0.30 | / | 0.06 | 0.13 | 0.04 |
| 65 | 0 | 21 | / | / | 21.71 | 1.38 | 1.11 | / | / | / |
| 65 | 11 | 2 | / | 0.73 | 23.77 | 0.29 | / | 0.07 | 0.11 | / |
| 65 | 11 | 17 | 1.47 | 1.09 | 16.55 | 0.35 | / | / | / | / |
| 70 | 11 | 1 | / | 0.74 | 23.41 | 0.29 | / | 0.06 | 0.09 | / |
| 70 | 11 | 10 | / | 1.10 | 17.67 | 0.47 | / | / | / | / |
| 75 | 0 | 1 | / | 0.61 | 25.10 | 0.30 | / | 0.06 | 0.09 | / |
| 75 | 0 | 7 | / | 1.43 | 25.13 | 2.60 | 1.42 | / | / | / |
| 75 | 11 | 1 | 1.84 | 0.74 | 22.27 | 0.24 | / | 0.07 | 0.12 | / |
| 75 | 11 | 6 | / | / | 18.09 | 1.27 | 0.73 | / | / | / |
| T/ | RH/ | Time/ | RRT | | | | | | | |
| ° C. | % | day | 0.75 | 0.78 | 0.84 | 0.87 | 1.00 | 1.05 | 1.15 | 1.21 |
| 0 | 0 | 0 | 0.68 | / | / | 0.10 | 70.89 | / | 1.59 | 0.03 |
| 0 | 0 | 0 | 0.70 | / | / | 0.10 | 69.21 | / | 1.56 | / |
| 55 | 0 | 10 | 0.69 | / | / | 0.10 | 68.66 | / | 1.70 | 0.10 |
| 55 | 0 | 21 | 0.71 | / | / | 0.12 | 70.97 | / | 1.72 | 0.15 |
| 55 | 11 | 7 | 0.59 | 0.14 | / | 0.10 | 71.05 | / | 1.69 | 0.06 |
| 55 | 11 | 21 | 0.74 | / | / | 0.11 | 70.13 | / | 1.84 | 0.30 |
| 65 | 0 | 3 | 0.71 | / | / | 0.10 | 70.62 | / | 1.89 | / |
| 65 | 0 | 21 | 0.85 | / | / | / | 58.15 | / | 3.80 | / |
| 65 | 11 | 2 | 0.59 | 0.16 | / | 0.10 | 71.97 | / | 1.80 | / |
| 65 | 11 | 17 | 0.90 | / | / | 0.14 | 64.43 | / | 3.40 | 0.27 |
| 70 | 11 | 1 | 0.59 | / | 0.15 | 0.10 | 72.32 | / | 1.80 | / |
| 70 | 11 | 10 | 0.92 | / | / | 0.12 | 66.05 | / | 3.44 | 0.25 |
| 75 | 0 | 1 | 0.71 | / | / | 0.10 | 69.93 | / | 1.91 | / |
| 75 | 0 | 7 | 0.65 | 0.15 | 0.07 | 0.19 | 50.65 | 3.52 | 4.09 | / |
| 75 | 11 | 1 | 0.56 | 0.14 | / | 0.09 | 68.85 | / | 1.83 | / |
| 75 | 11 | 6 | 0.87 | / | / | 0.14 | 64.80 | / | 2.82 | 0.16 |

| T/ | RH/ | Time/ | RRT | | | | | | | | |
|---|---|---|---|---|---|---|---|---|---|---|---|
| ° C. | % | day | 1.36 | 1.44 | 1.57 | 1.71 | 1.83 | 1.89 | 2.09 | 2.46 | 2.51 |
| 0 | 0 | 0 | / | / | / | / | / | / | / | / | / |
| 0 | 0 | 0 | 1.78 | / | / | / | / | / | / | / | / |
| 55 | 0 | 10 | / | / | 2.21 | / | / | 0.18 | 1.21 | / | / |
| 55 | 0 | 21 | 2.30 | / | 0.71 | / | / | 0.26 | 0.45 | / | / |
| 55 | 11 | 7 | / | / | 0.83 | / | / | 0.19 | 0.45 | / | / |
| 55 | 11 | 21 | 1.88 | / | 2.77 | / | / | 0.39 | 1.62 | / | / |
| 65 | 0 | 3 | / | / | 0.72 | / | / | 0.18 | 0.42 | / | / |
| 65 | 0 | 21 | / | 0.20 | 6.16 | 0.17 | 0.18 | 1.20 | 4.54 | 0.55 | / |
| 65 | 11 | 2 | / | / | 0.26 | / | / | / | 0.17 | / | / |
| 65 | 11 | 17 | 1.17 | / | 5.48 | 0.18 | 0.10 | 1.18 | 3.30 | / | / |
| 70 | 11 | 1 | / | / | 0.29 | / | / | / | 0.17 | / | / |
| 70 | 11 | 10 | 0.09 | 0.14 | 5.16 | 0.15 | 0.14 | 1.00 | 3.28 | / | / |
| 75 | 0 | 1 | / | / | 0.77 | / | / | / | 0.41 | / | / |
| 75 | 0 | 7 | / | 0.16 | 6.32 | 0.14 | / | 0.48 | 2.90 | 0.11 | / |
| 75 | 11 | 1 | 2.67 | / | 0.40 | / | / | / | 0.18 | / | / |
| 75 | 11 | 6 | / | 0.13 | 6.34 | 0.10 | 0.11 | 0.97 | 3.15 | 0.09 | 0.25 |

TABLE 12
Test result of related substance of composition A14

| T/ | RH/ | Time/ | RRT | | | | | |
|---|---|---|---|---|---|---|---|---|
| ° C. | % | day | 0.12 | 0.17 | 0.21 | 0.27 | 0.29 | 0.31 |
| 0 | 0 | 0 | / | / | 0.17 | 0.31 | / | / |
| 0 | 0 | 0 | / | 0.71 | / | 0.41 | / | / |
| 55 | 0 | 10 | / | / | 0.34 | 0.47 | / | 0.09 |
| 55 | 0 | 21 | 0.69 | / | 0.57 | 0.74 | 0.18 | / |
| 55 | 11 | 7 | / | / | 0.31 | 0.43 | / | 0.11 |
| 55 | 11 | 21 | 0.69 | / | 0.44 | 0.67 | / | 0.20 |
| 65 | 0 | 3 | / | / | 0.33 | 0.45 | / | 0.09 |
| 65 | 0 | 21 | / | / | 0.86 | 0.72 | 0.20 | / |
| 65 | 11 | 2 | / | / | 0.34 | 0.41 | / | 0.10 |
| 65 | 11 | 17 | / | 0.41 | 0.89 | 0.79 | / | 0.25 |
| 70 | 11 | 1 | / | / | 0.34 | 0.44 | / | 0.10 |
| 70 | 11 | 10 | / | / | 0.64 | 0.86 | / | 0.23 |
| 75 | 0 | 1 | / | / | 0.33 | 0.43 | / | 0.08 |
| 75 | 0 | 7 | / | / | 0.99 | 0.89 | / | / |
| 75 | 11 | 1 | / | 1.29 | / | 0.43 | / | 0.15 |
| 75 | 11 | 6 | / | / | 0.73 | 0.77 | / | 0.15 |
| T/ | RH/ | Time/ | RRT | | | | | |
| ° C. | % | day | 0.32 | 0.63 | 0.75 | 0.78 | 0.88 | 1.00 |
| 0 | 0 | 0 | 0.24 | 0.08 | 0.78 | 0.19 | 0.13 | 95.54 |
| 0 | 0 | 0 | 0.35 | 0.08 | 0.75 | 0.20 | 0.13 | 94.87 |
| 55 | 0 | 10 | 0.24 | 0.07 | 0.99 | / | 0.14 | 94.74 |
| 55 | 0 | 21 | 0.21 | 0.08 | 0.81 | 0.20 | 0.19 | 92.50 |
| 55 | 11 | 7 | 0.28 | 0.08 | 0.79 | 0.20 | 0.14 | 95.67 |
| 55 | 11 | 21 | 0.23 | 0.08 | 0.80 | 0.20 | 0.17 | 92.93 |
| 65 | 0 | 3 | 0.23 | 0.08 | 0.79 | 0.21 | 0.14 | 94.91 |
| 65 | 0 | 21 | 0.25 | 0.07 | 0.86 | 0.22 | 0.23 | 90.34 |
| 65 | 11 | 2 | 0.28 | 0.08 | 0.79 | 0.20 | 0.14 | 95.07 |
| 65 | 11 | 17 | 0.45 | 0.07 | 1.08 | / | 0.22 | 89.65 |
| 70 | 11 | 1 | 0.27 | 0.08 | 0.97 | / | 0.14 | 95.03 |
| 70 | 11 | 10 | 0.31 | 0.07 | 1.08 | / | 0.22 | 89.10 |
| 75 | 0 | 1 | 0.25 | 0.07 | 0.78 | 0.19 | 0.13 | 94.95 |
| 75 | 0 | 7 | 0.32 | 0.07 | 1.13 | / | 0.24 | 89.48 |
| 75 | 11 | 1 | 0.31 | 0.08 | 0.98 | / | 0.14 | 94.02 |
| 75 | 11 | 6 | 0.15 | 0.07 | 0.88 | 0.20 | 0.24 | 89.23 |

| T/ | RH/ | Time/ | RRT | | | | | | |
|---|---|---|---|---|---|---|---|---|---|
| ° C. | % | day | 1.16 | 1.26 | 1.44 | 1.57 | 1.89 | 2.10 | 2.46 |
| 0 | 0 | 0 | 2.49 | 0.08 | / | / | / | / | / |
| 0 | 0 | 0 | 2.42 | 0.07 | / | / | / | / | / |
| 55 | 0 | 10 | 2.56 | / | / | 0.36 | / | / | / |
| 55 | 0 | 21 | 2.73 | / | / | 0.99 | / | 0.12 | / |
| 55 | 11 | 7 | 2.59 | 0.06 | / | 0.09 | / | / | / |
| 55 | 11 | 21 | 2.75 | / | / | 0.83 | / | / | / |
| 65 | 0 | 3 | 2.61 | / | / | 0.16 | / | / | / |
| 65 | 0 | 21 | 3.02 | / | 0.09 | 2.31 | 0.17 | 0.54 | 0.12 |
| 65 | 11 | 2 | 2.55 | 0.06 | / | / | / | / | / |
| 65 | 11 | 17 | 2.97 | / | 0.06 | 2.40 | 0.25 | 0.54 | / |
| 70 | 11 | 1 | 2.57 | 0.05 | / | / | / | / | / |
| 70 | 11 | 10 | 3.25 | / | 0.12 | 2.75 | 0.45 | 0.91 | / |
| 75 | 0 | 1 | 2.59 | / | / | 0.19 | / | / | / |
| 75 | 0 | 7 | 3.09 | 0.05 | 0.11 | 2.88 | / | 0.57 | 0.18 |
| 75 | 11 | 1 | 2.61 | / | / | / | / | / | / |
| 75 | 11 | 6 | 3.17 | / | 0.10 | 3.48 | 0.31 | 0.52 | / |

TABLE 13
Test result of related substance of API raw material

| T/ | RH/ | Time/ | RRT | | | | | |
|---|---|---|---|---|---|---|---|---|
| ° C. | % | day | 0.21 | 0.27 | 0.30 | 0.32 | 0.38 | 0.48 |
| 0 | 0 | 0 | / | 0.21 | / | 0.34 | / | / |
| 0 | 0 | 0 | / | 0.14 | / | 0.31 | / | / |
| 55 | 0 | 10 | / | 0.23 | 0.08 | 0.24 | / | / |
| 55 | 0 | 21 | / | 0.47 | / | 0.23 | / | / |
| 55 | 11 | 7 | / | 0.25 | 0.10 | 0.24 | / | / |
| 55 | 11 | 21 | / | 0.59 | / | 0.21 | / | / |
| 65 | 0 | 3 | / | 0.23 | 0.05 | 0.23 | / | / |
| 65 | 0 | 21 | / | 0.24 | / | 0.18 | / | / |
| 65 | 11 | 2 | / | 0.18 | / | 0.22 | / | / |
| 65 | 11 | 17 | / | 0.44 | 0.14 | 0.17 | / | / |
| 70 | 11 | 1 | / | 0.25 | 0.06 | 0.24 | / | / |
| 70 | 11 | 10 | / | 0.39 | 0.19 | 0.22 | / | / |
| 75 | 0 | 1 | / | 0.25 | 0.05 | 0.23 | / | / |
| 75 | 0 | 7 | / | 0.37 | 0.15 | 0.24 | / | / |
| 75 | 11 | 1 | 0.55 | 0.26 | 0.05 | 0.20 | / | / |
| 75 | 11 | 6 | / | 0.37 | / | 0.25 | 0.05 | 0.02 |

| T/ | RH/ | Time/ | RRT | | | | | | |
|---|---|---|---|---|---|---|---|---|---|
| ° C. | % | day | 0.63 | 0.75 | 0.88 | 1.00 | 1.16 | 1.28 | 1.46 |
| 0 | 0 | 0 | 0.08 | 0.95 | 0.15 | 95.73 | 2.12 | 0.08 | / |
| 0 | 0 | 0 | 0.08 | 0.98 | 0.15 | 96.14 | 2.11 | 0.10 | / |
| 55 | 0 | 10 | 0.08 | 0.96 | 0.19 | 95.12 | 2.46 | 0.07 | / |
| 55 | 0 | 21 | 0.07 | 0.99 | 0.25 | 92.81 | 2.77 | / | 0.13 |
| 55 | 11 | 7 | 0.08 | 0.95 | 0.16 | 95.65 | 2.49 | 0.07 | / |
| 55 | 11 | 21 | 0.07 | 0.98 | 0.18 | 95.01 | 2.46 | 0.06 | / |
| 65 | 0 | 3 | 0.08 | 0.93 | 0.17 | 93.88 | 2.42 | 0.08 | / |
| 65 | 0 | 21 | 0.08 | 1.04 | 0.23 | 94.17 | 2.43 | 0.05 | 0.05 |
| 65 | 11 | 2 | 0.08 | 0.96 | 0.18 | 95.80 | 2.51 | 0.06 | / |
| 65 | 11 | 17 | 0.07 | 1.10 | 0.29 | 91.93 | 2.52 | / | 0.30 |
| 70 | 11 | 1 | 0.08 | 0.95 | 0.18 | 95.19 | 2.48 | 0.07 | / |
| 70 | 11 | 10 | 0.07 | 1.09 | 0.31 | 91.46 | 2.59 | / | 0.22 |
| 75 | 0 | 1 | 0.08 | 0.93 | 0.17 | 95.27 | 2.48 | 0.06 | / |
| 75 | 0 | 7 | 0.06 | 1.05 | 0.36 | 89.65 | 2.85 | / | 0.27 |
| 75 | 11 | 1 | 0.08 | 0.97 | 0.18 | 94.75 | 2.53 | 0.12 | 0.31 |
| 75 | 11 | 6 | 0.07 | 1.12 | 0.22 | 94.60 | 2.50 | / | / |

| T/ | RH/ | Time/ | RRT | | | | | |
|---|---|---|---|---|---|---|---|---|
| ° C. | % | day | 1.58 | 1.83 | 1.91 | 2.08 | 2.12 | 2.48 |
| 0 | 0 | 0 | / | / | / | 0.34 | / | / |
| 0 | 0 | 0 | / | / | / | / | / | / |
| 55 | 0 | 10 | 0.41 | / | / | 0.16 | / | / |
| 55 | 0 | 21 | 1.45 | 0.05 | 0.16 | / | 0.62 | / |
| 55 | 11 | 7 | / | / | / | / | / | / |
| 55 | 11 | 21 | 0.28 | / | 0.16 | / | / | / |
| 65 | 0 | 3 | 0.12 | / | / | 1.80 | / | / |
| 65 | 0 | 21 | 1.21 | / | 0.32 | / | / | / |
| 65 | 11 | 2 | / | / | / | / | / | / |
| 65 | 11 | 17 | 1.43 | / | 0.61 | / | 1.00 | / |
| 70 | 11 | 1 | / | / | / | 0.51 | / | / |
| 70 | 11 | 10 | 1.42 | / | 0.57 | / | 1.47 | / |
| 75 | 0 | 1 | 0.15 | / | / | 0.33 | / | / |
| 75 | 0 | 7 | 2.55 | / | 0.28 | / | 1.75 | 0.43 |
| 75 | 11 | 1 | / | / | / | / | / | / |
| 75 | 11 | 6 | / | 0.06 | 0.74 | / | / | / |

TABLE 14
Comparison of the purity results of the compositions
prepared in embodiments 1 to 12 and API

| T/ | RH/ | Time/ | | | | | | | |
|---|---|---|---|---|---|---|---|---|---|
| ° C. | % | day | A1/% | A2/% | A3/% | A5/% | A6/% | A7/% | API/% |
| 0 | 0 | 0 | 95.57 | 95.86 | 95.67 | 95.87 | 95.79 | 95.94 | 95.73 |
| 0 | 0 | 0 | 95.47 | 95.89 | 95.67 | 95.9 | 95.05 | 95.94 | 96.14 |
| 55 | 0 | 10 | 94.68 | 95.41 | 94.83 | 95.3 | 94.55 | 95.82 | 95.12 |
| 55 | 0 | 21 | 95.19 | 94.8 | 94.3 | 94.96 | 80.72 | 95.6 | 92.81 |
| 55 | 11 | 7 | 94.99 | 95.75 | 95.59 | 96.22 | 92.44 | 95.88 | 95.65 |
| 55 | 11 | 21 | 94.34 | 95.18 | 94.76 | 95.11 | 81.53 | 95.87 | 95.01 |
| 65 | 0 | 3 | 94.5 | 95.28 | 94.77 | 95.28 | 89.42 | 95.82 | 93.88 |
| 65 | 0 | 21 | 93.04 | 94.62 | 93 | 93.98 | 64.63 | 95.4 | 94.17 |
| 65 | 11 | 2 | 94.87 | 95.41 | 95.13 | 95.37 | 89.53 | 95.9 | 95.8 |
| 65 | 11 | 17 | 93.36 | 94.61 | 93.11 | 92.98 | 52.32 | 95.52 | 91.93 |
| 70 | 11 | 1 | 94.96 | 95.41 | 94.98 | 95.04 | 86.73 | 95.86 | 95.19 |
| 70 | 11 | 10 | 92.51 | 94.45 | 92.57 | 89.73 | 19.33 | 95.35 | 91.46 |
| 75 | 0 | 1 | 94.8 | 95.39 | 94.58 | 95.13 | 81.86 | 95.82 | 95.27 |
| 75 | 0 | 7 | 92.19 | 94.07 | 93.18 | 93.97 | 13.84 | 95.25 | 89.65 |
| 75 | 11 | 1 | 94.56 | 95.32 | 94.43 | 93.55 | 78.23 | 95.83 | 94.75 |
| 75 | 11 | 6 | 92.84 | 93.93 | 91.61 | 84.65 | 18.06 | 95.36 | 94.6 |

| T/ | RH/ | Time/ | | | | | | |
|---|---|---|---|---|---|---|---|---|
| ° C. | % | day | A9/% | A11/% | A12/% | A13/% | A14/% | API/% |
| 0 | 0 | 0 | 95.81 | 74.61 | 94.84 | 70.89 | 95.54 | 95.73 |
| 0 | 0 | 0 | 95.81 | 74.48 | 93.28 | 69.21 | 94.87 | 96.14 |
| 55 | 0 | 10 | 93.36 | 70.73 | 92.08 | 68.66 | 94.74 | 95.12 |
| 55 | 0 | 21 | 88.66 | 70.73 | 90.59 | 70.97 | 92.5 | 92.81 |
| 55 | 11 | 7 | 94.92 | 74.25 | 93.59 | 71.05 | 95.67 | 95.65 |
| 55 | 11 | 21 | 93.47 | 72.6 | 86.68 | 70.13 | 92.93 | 95.01 |
| 65 | 0 | 3 | 93.51 | 73.91 | 93.48 | 70.62 | 94.91 | 93.88 |
| 65 | 0 | 21 | 88.95 | 61.54 | 76.87 | 58.15 | 90.34 | 94.17 |
| 65 | 11 | 2 | 94.22 | 74.16 | 94.08 | 71.97 | 95.07 | 95.8 |
| 65 | 11 | 17 | 86.84 | 67.18 | 79.64 | 64.43 | 89.65 | 91.93 |
| 70 | 11 | 1 | 94.49 | 74.31 | 94.07 | 72.32 | 95.03 | 95.19 |
| 70 | 11 | 10 | 84.42 | 65.8 | 79.28 | 66.05 | 89.1 | 91.46 |
| 75 | 0 | 1 | 93.76 | 77.32 | 93.36 | 69.93 | 94.95 | 95.27 |
| 75 | 0 | 7 | 84.71 | 63.80 | 73.31 | 50.65 | 89.48 | 89.65 |
| 75 | 11 | 1 | 93.21 | 73.46 | 89.21 | 68.85 | 94.02 | 94.75 |
| 75 | 11 | 6 | 83.36 | 63.8 | 76.9 | 64.8 | 89.23 | 94.6 |

Referring to tables 1 to 14, RRT (relative retention time) denotes a ratio of the retention time of the corresponding substance to the retention time of the API (the angiotensin II). The results shown in the tables were obtained using area normalization. As shown in tables 1 to 14, the test of the composition were performed under the test temperature (T, 0° C., 55° C., 65° C., 70° C., 75° C., respectively) and the test relative humidity (RH, 0% and 11% respectively). After the test time, the purity of the angiotensin II in the compositions prepared by the embodiment 1 to the embodiment 12 and the API were detected. The results show that the compositions A2, A7, and A10 may significantly reduce the increase of impurities and/or reduce the variation of the content of the angiotensin II compared with other compositions and the API.

### Embodiment 13—Stability Test

For the compositions A2, A7, and A10, the ASAP Prime® (Accelerated Stability Assessment Program) was used to determine the product shelf-life of the above three compositions. The ASAP Prime® is a scientific approach that can accurately predict long-term stability in a short period of time based on accelerated stability data. Based on the physical and chemical properties of the material, it can design a reasonable stressing experiment (for example, combinations of different temperatures, humidity, and time) that content or the amount(s) of impurities are measured after a period of time, followed by degradation kinetics modeling and shelf-life prediction. The principle of ASAP Prime® can be characterized by the humidity-corrected Arrhenius equation, as follows:

$$ln ( k ) = ln ( A ) - \frac{E a}{R T} + B ( RH )$$

wherein k denotes the rate at which the angiotensin II content decreases or the rate at which the impurities increase (may also be calculated by the specification level/isoconversion time) when the temperature is T, A is the Arrhenius constant (also called collision frequency), Ea is the activation energy (unit is kcal/mol) of degradation, R is the ideal gas constant, T is the absolute temperature (unit is K), and B is the humidity sensitivity factor. The shelf-life prediction results of the compositions A2, A7, and A10 as shown in table. The apparently varying impurities were respectively: an impurity of RRT of 1.59 in the composition A2, impurities of RRT of 0.57 and 0.62 in the composition A7, and impurities of RRT of 1.19 and 1.60 in the composition A10. The growth limit value of the specified impurity is 0.5% (for example, the increment in the impurity that is less than 0.5% is stable), and the predicted results are shown in Table 15:

TABLE 15
Average time for the growth of the composition
impurity to reach the limit value

| Composition | Impurity | 25° C. | 30° C. |
|---|---|---|---|
| A2 | RRT1.59 | >3 years | >3 years |
| A7 | RRT0.57 | >3 years | >3 years |
| | RRT0.62 | >3 years | >3 years |
| A10 | RRT1.19 | >3 years | 2 years |
| | RRT1.60 | >3 years | >3 years |

According to Table 15, it may be concluded that the storage stability of the composition A2 under 25° C. and 30° C. are greater than 3 years, and the storage stability of the composition A7 under 25° C. and 30° C. are also greater than 3 years, and the storage stability of the composition A10 under 25° C. and 30° C. are 2 years. The present disclosure may increase the stability of the composition including the angiotensin II, possibly extending its shelf-life. The present disclosure may be stored under room temperature, possibly reducing its storage requirements.

The benefit of the present disclosure for the stable composition including the angiotensin II may include but be not limited to: the composition has high stability and may be stored at room temperature for at least two years, so it may extend the shelf-life of the angiotensin II, which may reduce the requirement for storage conditions of the angiotensin II. The benefit of the present disclosure for the preparation method of the stable composition including the angiotensin II may include but be not limited to: the preparation process is simple, easy to handle, and does not damage or reduce the active ingredient of the angiotensin II. The benefit of the present disclosure for the method for using the stable composition including the angiotensin II may include but be not limited to: when used, it may be used by direct squeezing the liquid chamber to reconstitute the composition, and may be used for rapid drug preparation, which is suitable for first aid.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Similarly, it should be appreciated that in the foregoing description of embodiments of the present disclosure, various features are sometimes grouped in a single embodiment, figure, or descriptions thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about”, “approximate”, or “substantially”. For example, “about”, “approximate” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A solid composition consisting of angiotensin II and trehalose, wherein a mass ratio of the angiotensin II to the trehalose is about 0.3:10.

2-11. (canceled)

12. A method for using the composition of claim 1, comprising:
storing the composition in a powder chamber of a dual chamber bag, and an isotonic solution for infusion in a liquid chamber of the dual chamber bag, wherein the powder chamber is isolated from the liquid chamber by a peelable seal; and squeezing the liquid chamber bag to detach the peelable seal to allow mixing of the composition and the isotonic solution for infusion usage.

13. A method for preparing a stable composition including angiotensin II, the composition further includes an excipient including one or more substances selected from a sugar, an alcohol, a polymer, a surfactant, or an amino acid, wherein the method comprises:
dissolving the angiotensin II and the excipient in a solvent to obtain a solution, wherein the solvent is selected from one or more of the following solvents: water, acetonitrile, N-methylpyrrolidone, dimethyl sulfoxide, dichloromethane, acetone, or alcohol; and
solidifying the solution to obtain the composition.

14. The method of claim 13, wherein a mass ratio of the angiotensin II to the excipient is no more than 0.3:10.

15. The method of claim 13, wherein after the composition was stored under a first environment temperature for a first time period after preparation, a reduction value of a purity of the angiotensin II is less than a first threshold, and an increment of a first impurity associated with the angiotensin II is less than a second threshold, the first time period is at least two years; the first threshold is 5%, and the second threshold is 0.5%.

16. The method of claim 15, wherein the first environment temperature is no more than 30° C.

17. A method for using a stable composition including angiotensin II, the composition further includes an excipient including one or more substances selected from a sugar, an alcohol, a polymer, a surfactant, or an amino acid, wherein the method comprises:
storing the composition in a medical container, the medical container at least including a sealed vial,
adding a medical liquid to the medical container to dissolve the composition; and
using the medical liquid in which the composition is dissolved as an infusion solution, or an injection solution.

18. The method of claim 17, wherein a mass ratio of the angiotensin II to the excipient is no more than 0.3:10.

19. The method of claim 17, wherein after the composition was stored under a first environment temperature for a first time period after preparation, a reduction value of a purity of the angiotensin II is less than a first threshold, and an increment of a first impurity associated with the angiotensin II is less than a second threshold, the first time period is at least two years; the first threshold is 5%, and the second threshold is 0.5%.

20. The method of claim 19, wherein the first environment temperature is no more than 30° C.

21. The composition of claim 1, after the composition is stored at a temperature of 55° C. for 7-21 days after preparation, a reduction value of a purity of the angiotensin II is less than 0.5%, measured by normalization of peak areas in a High Performance Liquid Chromatography (HPLC) assay.

22. The composition of claim 1, wherein when the composition is stored at the temperature of 65° C. for 3-17 days after preparation, a reduction value of a purity of the angiotensin II is less than 0.5%, measured by normalization of peak areas in a High Performance Liquid Chromatography (HPLC) assay.

23. The composition of claim 22, wherein a relative humidity for storage is about 0-11%.
